(19) United States
(12) Patent Application Publication
(10) Pub. No.: US 2024/0102034 A1
(43) Pub. Date: Mar. 28, 2024

(54) CANNABIS PLANT WITH INCREASED CANNABIGEROLIC ACID

(71) Applicants: Daniel Kevin FOWLER, Arvada, CO (US); CANOPY GROWTH COPORATION

(72) Inventor: Daniel Kevin FOWLER, Arvada, CO (US)

(21) Appl. No.: 17/641,985

(22) PCT Filed: Jul. 15, 2021
(86) PCT No.: PCT/US21/41818

§ 371 Date: Mar. 10, 2022

Related U.S. Application Data
(60) Provisional application No. 63/052,817, filed on Jul. 16, 2020.
(60) Provisional application No. 63/053,413, filed on Jul. 17, 2020.
(60) Provisional application No. 63/196,981, filed on Jun. 4, 2021.

(51) Int. Cl. C12N 15/82; A01H 6/28; C12N 9/02; C12N 9/22

(52) U.S. Cl. CPC C12N 15/8243; A01H 6/28; C12N 9/0004; C12N 9/22; C12N 15/8213; C12N 2310/20; C12Y 121/03007

(57) ABSTRACT

The present technology generally relates to Cannabis plants having increased cannabigerolic acid (CBGA) and/or cannabigerol (CBG) content. The present technology also generally relates to isolated nucleic acids and isolated amino acids related to same and methods of producing same.

### CROSS-REFERENCE TO RELATED APPLICATIONS

This Application claims the benefit of and priority to U.S. provisional patent application No. 63/052,817, filed on Jul. 16, 2020; to U.S. provisional patent application No. 63/053,413, filed on Jul. 17, 2020; to U.S. provisional patent application No. 63/196,981, filed on Jun. 4, 2021, the content of all of is herein incorporated in entirety by reference.

### FIELD OF TECHNOLOGY

The present technology generally relates to Cannabis plants having increased cannabigerolic acid (CBGA) and/or cannabigerol (CBG) content as well as to nucleic acids related to same and methods of producing same.

### BACKGROUND INFORMATION

Cannabis is a genus of flowering plants that produce a unique class of terpenophenolic compounds known as cannabinoids. Cannabinoids interact with receptors of human and animal endocannabinoid systems and can lead to a plethora of potential medical and therapeutic effects (Di Marzo & Piscitelli, 2015). In the Cannabis plant's biosynthetic pathway, cannabigerolic acid (CBGA) is the first cannabinoid produced by enzymatic condensation of olivetolic acid and geranyl pyrophosphate (Gagne et al., 2012). CBGA is then converted to Δ⁹-tetrahydrocannabinolic acid (THCA) and cannabidiolic acid (CBDA) by the enzymes THCA synthase (THCAS) and CBDA synthase (CBDAS), respectively (Laverty et al., 2019) (FIG. 1). Non-enzymatic decarboxylation of the cannabinoids, primarily through heating, produces the neutral forms: cannabigerol (CBG), Δ⁹-tetrahydrocannabinol (THC) and cannabidiol (CBD) (FIG. 1).

THC, the major intoxicating cannabinoid responsible for Cannabis' “high” when inhaled or ingested, and CBD, a non-intoxicating cannabinoid are the two most prevalent cannabinoids in individual Cannabis cultivars, known colloquially as “strains,” and have been extensively studied for human and animal health purposes (Lewis et al., 2018). However over 70 other less abundant cannabinoids have been found in Cannabis plant samples, many of which have promising pharmacological activity (ElSohly & Slade, 2005). CBG (produced as CBGA in planta), typically found in trace amounts in most Cannabis strains, is one such cannabinoid.

Recent studies have suggested multiple medical uses for CBG. Rodent models show CBG is an appetite stimulant (Brierley et al., 2016), inhibits colon carcinogenesis (Borrelli et al., 2014), prevents colitis in a model of inflammatory bowel disease (Borrelli et al., 2013), decreases intraocular pressure associated with glaucoma (Colasanti et al., 1984) and is neuroprotective in a model of Huntington's disease (Valdeolivas et al., 2015). Additional cellular studies suggest CBG can prevent neuroinflammation (Gugliandolo et al., 2018).

Because it is typically a rare cannabinoid, there is a need to develop Cannabis strains which produce higher cannabigerolic acid (CBGA) and/or cannabigerol (CBG) levels.

### SUMMARY OF DISCLOSURE

According to various aspects, the present technology relates to a Cannabis plant, plant part, tissue or cell thereof, wherein the Cannabis plant, plant part, tissue or cell thereof has a cannabigerol (CBG) content greater than about 5% by weight.

According to various aspects, the present technology relates to a Cannabis plant, plant part, tissue or cell thereof, wherein the Cannabis plant, plant part, tissue or cell thereof has a cannabigerol (CBG) content greater than about 5%, 6%, 7%, 8%, 9%, 10%, 11%, 12%, 13%, 14% or 15% by weight.

According to various aspects, the present technology relates to a Cannabis plant, plant part, tissue or cell thereof, wherein the Cannabis plant, plant part, tissue or cell thereof has a cannabigerol (CBG) content greater than about 10% by weight.

According to various aspects, the present technology relates to a Cannabis plant, plant part, tissue or cell thereof, wherein the Cannabis plant, plant part, tissue or cell thereof has a cannabigerol (CBG) content of between about 5% and about 25% by weight.

According to various aspects, the present technology relates to a Cannabis plant, plant part, tissue or cell thereof, wherein the Cannabis plant, plant part, tissue or cell thereof has a cannabigerol (CBG) content of between about 5% and about 15% by weight.

According to various aspects, the present technology relates to a Cannabis plant, plant part, tissue or cell thereof, wherein the Cannabis plant, plant part, tissue or cell thereof has a cannabigerol (CBG) content of between about 5% and about 10% by weight.

According to various aspects, the present technology relates to an isolated nucleic acid molecule comprising an altered tetrahydrocannabinolic acid synthase (THCAS) allele, wherein the altered THCAS allele encodes for a minimally functional or non-functional THCAS and causes an increase in cannabigerolic acid (CBGA) and/or cannabigerol (CBG) production. In some implementations of these aspects, the altered THCAS allele encodes for a minimally functional or non-functional THCAS that impedes conversion of CBGA to THCA. In some instances, the minimally functional or the non-functional THCAS comprises an activity-altering change.

According to various aspects, the present technology relates to an isolated nucleic acid molecule comprising a nucleotide sequence having at least greater than or about 75% sequence identity to SEQ ID NO: 2, wherein the nucleic acid sequence comprises the activity-altering change that encodes for a minimally functional or non-functional tetrahydrocannabinolic acid synthase (THCAS).

According to various aspects, the present technology relates to an isolated nucleic acid molecule comprising a cDNA having a nucleotide sequence having at least, greater than or about 75%, or greater than or about 85% sequence identity to the complementary sequence of SEQ ID NO: 2, wherein the cDNA encodes for a minimally functional or a non-functional tetrahydrocannabinolic acid synthase (THCAS).

According to various aspects, the present technology relates to an isolated DNA marker for identifying a minimally functional or a non-functional tetrahydrocannabinolic acid synthase (THCAS) in a plant exhibiting THCAS activity, the isolated DNA marker comprising SEQ ID NO: 2 or a fragment thereof comprising an activity-altering change.

According to various aspects, the present technology relates to an isolated polypeptide encoded by the isolated nucleic acid molecule as defined herein.

According to various aspects, the present technology relates to an antibody that specifically binds the isolated polypeptide defined herein.

According to various aspects, the present technology relates to an organism, tissue or cell comprising the isolated nucleic molecule or the isolates polypeptides or the expression systems as defined herein. In some instances, the organism, tissue of cell is a plant, a plant tissue, or a plant cell. In some further instances, the organism, tissue of cell is a Cannabis plant, a Cannabis tissue, or a Cannabis cell.

According to various aspects, the present technology relates to a Cannabis plant comprising an altered tetrahydrocannabinolic acid (THCA) synthase allele, wherein the altered tetrahydrocannabinolic acid (THCA) synthase allele encodes a minimally functional or a non-functional tetrahydrocannabinolic acid (THCA) synthase that causes an increase in cannabigerolic acid (CBGA) and/or cannabigerol (CBG) levels. In some instances, the alteration is an activity-altering mutation.

According to various aspects, the present technology relates to a Cannabis plant comprising an altered tetrahydrocannabinolic acid synthase (THCAS) allele, wherein the altered tetrahydrocannabinolic acid (THCA) synthase allele prevents conversion of cannabigerolic acid (CBGA) to tetrahydrocannabinolic acid (THCA). In some instances, the alteration results in a minimally-functional or a non-functional THCAs enzyme.

According to various aspects, the present technology relates to a method for increasing levels of cannabigerolic acid (CBGA) and/or cannabigerol (CBG) in a tissue or a cell, the method comprising introducing an activity-altering change in the nucleic acid sequence encoding for tetrahydrocannabinolic acid synthase (THCAS) in the tissue or the cell. In some instances, the activity-altering change results in a THCAS allele that encodes for a minimally functional or non-functional THCAS that impedes conversion of CBGA to THCA.

According to various aspects, the present technology relates to a method of increasing levels of cannabigerolic acid (CBGA) and/or cannabigerol (CBG) in a tissue or a cell, the method comprising: a) introducing into a tissue or a cell producing tetrahydrocannabinolic acid (THCA) synthase, a vector comprising: i) a nucleic acid sequence comprising SEQ ID NO: 2 or a fragment thereof, the fragment thereof retaining an activity-altering change; or ii) a nucleic acid sequence having at least or about 75% sequence identity to SEQ ID NO: 2 while retaining the an activity-altering change; to produce a recombinant tissue or recombinant cell; b) culturing the recombinant tissue or the recombinant cell under conditions that permit expression of the nucleic acid.

According to various aspects, the present technology relates to a method comprising the steps of: detecting the presence of a tetrahydrocannabinolic acid synthase (THCAS) gene sequence comprising a THCA synthase gene sequence in a sample that comprises nucleic acid from a Cannabis plant, and detecting the presence of an activity-altered THCAS gene sequence in a sample that comprises nucleic acid from the Cannabis plant, wherein the activity-altered THCAS gene sequence includes at least one activity-altering change that alters the activity of a THCAS encoded by the activity-altered THCAS gene sequence relative to a THCAS encoded by a wild-type THCAS gene sequence.

Other aspects and features of the present disclosure will become apparent to those ordinarily skilled in the art upon review of the following description of specific embodiments.

### BRIEF DESCRIPTION OF THE DRAWINGS

All features of embodiments which are described in this disclosure are not mutually exclusive and can be combined with one another. For example, elements of one embodiment can be utilized in the other embodiments without further mention. A detailed description of specific embodiments is provided herein below with reference to the accompanying drawings in which:

FIG. 1 is a diagram showing the biosynthesis pathway for cannabigerolic acid (CBGA) tetrahydrocannabinolic acid (THCA) and cannabidiolic acid (CBDA).

FIG. 2 is a photograph of a gel showing the results of a PCR assay used to amplify. THCA synthase (THCAS) and CBDA synthase (CBDAS) alleles from genomic DNA (gDNA) of a CBGA-dominant Cannabis strain and a THCA-dominant medicinal Cannabis strain Balmoral®.

FIG. 3 is a THCA synthase (THCAS) nucleic acid sequence alignment. THCAS nucleic acid sequence from a CBGA-dominant Cannabis plant showing the single nucleotide polymorphism (SNP) G1064A (THCAS^{G1064A}) when aligned against the THCAS nucleic acid sequence of Balmoral®, a THCA-dominant medicinal Cannabis strain.

FIG. 4 is a THCAS amino acid sequence alignment. Translated THCAS^{G1064A} sequence aligned against translated THCAS sequence from Balmoral®. G1064A SNP leads to an amino acid change at position 355 where an asparagine is present in place of a serine (S355N).

FIG. 5 shows the Cannabinoid profile of homozygous THCAS^{G1064A} Cannabis plant at 7 weeks post flowering.

FIG. 6 is a diagram of the tetra-primer allele refractory mutation system PCR to discriminate between THCAS^{1064G} and THCAS^{1064A} alleles according to one embodiment of the present technology.

FIG. 7 is a photograph of a gel showing the results of a PCR assay discriminating between wild type THCAS allele and a mutant THCAS according to one embodiment of the present technology. Tetra-primer allele refractory mutation system PCR on genomic DNA isolated from Cannabis homozygous for wild-type THCAS (THCAS^{1064G/1064G}), homozygous for mutant THCAS (THCAS^{1064/1064A}) and heterozygous for both alleles (THCAS^{1064G/1064A}).

FIG. 8 is a photograph of a gel showing the results of a PCR assay used to amplify THCAS and CBDAS alleles from gDNA of a CBGA-dominant parent plant homozygous for mutant THCAS (THCAS^{1064A/1064A}), a CBDA-dominant parent plant, and 15 of their F1 progeny.

FIG. 9 is a photograph of a gel showing the results of a PCR assay discriminating between wild type THCAS allele and a mutant THCAS according to one embodiment of the present technology. Tetra-primer allele refractory mutation system PCR on gDNA isolated from F1 progeny from a CBGA-dominant and CBDA-dominant plant cross, and the CBGA-dominant parent homozygous for mutant THCAS (THCAS^{1064A/1064A}). Genomic DNA from THCA-dominant plant Balmoral© homozygous for the wild type THCAS (THCAS^{1064G/1064G}) allele was used as a control.

FIG. 10 shows the total CBG content, total CBD content, and total CBG:CBD ratios of flowers from 12 F1 progeny of a CBGA-dominant parent homozygous for mutant THCAS (THCAS^{1064A/1064A}) and a CBDA-dominant parent, and 2 plants of each parent at 7 weeks post flowering.

FIG. 11 shows the total THC content, and total CBG:THC ratios of flowers from 12 F1 progeny of a CBGA-dominant parent homozygous for mutant THCAS (THCAS^{1064A/1064A}) and a CBDA-dominant parent, and 2 plants of each parent at 7 weeks post flowering.

FIG. 12 is a photograph of a gel showing the results of a PCR assay used to amplify THCAS and CBDAS alleles from gDNA of a CBDA-dominant F1 parent plant heterozygous for mutant THCAS (THCAS^{1064A}) and CBDAS, and 16 of its selfed (S1) progeny. The letter under the S1 progeny number indicates the inferred THCAS and CBDAS allelic composition; T=THCAS homozygote, H=THCAS/CBDAS heterozygote, C=CBDAS homozygote.

FIG. 13 is a photograph of a gel showing the results of a PCR assay discriminating between wild type THCAS allele and a mutant THCAS according to one embodiment of the present technology. Tetra-primer allele refractory mutation system PCR on gDNA isolated from a CBDA-dominant F1 parent plant heterozygous for mutant THCAS (THCAS^{1064A}) and CBDAS, and 16 of its selfed (S1) progeny. Genomic DNA from THCA-dominant plant Balmoral® homozygous for the wild type THCAS (THCAS^{1064G/1064G}) allele was used as a control.

FIG. 14 shows the total CBG content, total CBD content, and total CBG:CBD ratios of flowers from 16 selfed (S1) progeny of a CBDA-dominant F1 parent plant heterozygous for mutant THCAS (THCAS^{1064A}) and CBDAS and 2 plants of the parent sampled early at 4 weeks post flowering. S1 progeny were categorized into THCAS homozygous, THCAS/CBDAS heterozygous, and CBDAS homozygous based allele screening results in FIG. 12.

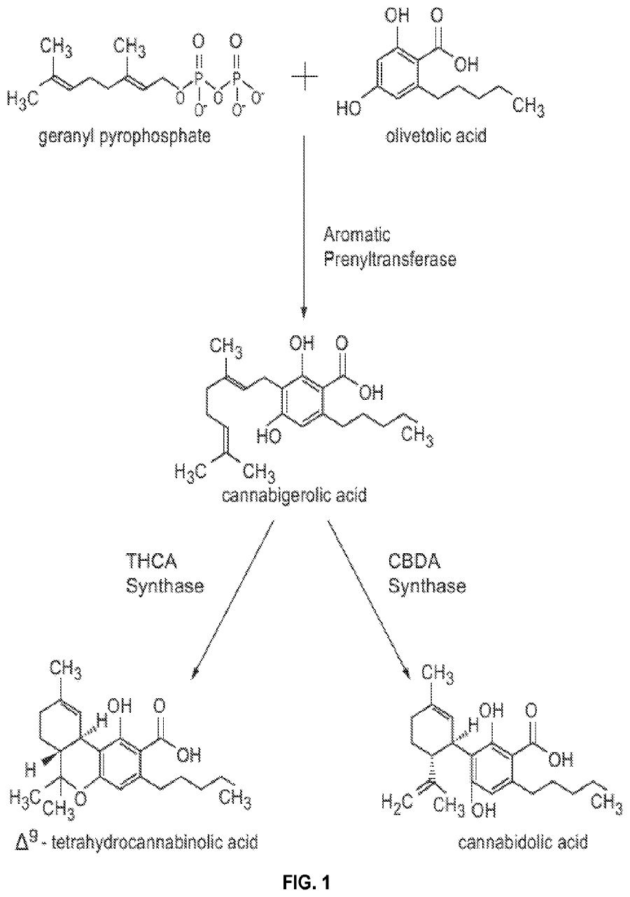

### DETAILED DISCLOSURE OF EMBODIMENTS

The present technology is explained in greater detail below. This description is not intended to be a detailed catalog of all the different ways in which the technology may be implemented, or all the features that may be added to the instant technology. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure in which variations and additions do not depart from the present technology. Hence, the following description is intended to illustrate some particular embodiments of the technology, and not to exhaustively specify all permutations, combinations and variations thereof.

As used herein, the singular form “a,” “an” and “the” include plural referents unless the context clearly dictates otherwise.

The recitation herein of numerical ranges by endpoints is intended to include all numbers subsumed within that range (e.g., a recitation of 1 to 5 includes 1, 1.25, 1.5, 1.75, 2, 2.45, 2.75, 3, 3.80, 4, 4.32, and 5).

The term “about” is used herein explicitly or not. Every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. For example, the term “about” in the context of a given value or range refers to a value or range that is within 20%, preferably within 15%, more preferably within 10%, more preferably within 9%, more preferably within 8%, more preferably within 7%, more preferably within 6%, and more preferably within 5% of the given value or range.

The expression “and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein. The term “or” as used herein should in general be construed non-exclusively. For example, an embodiment of “a composition comprising A or B” would typically present an aspect with a composition comprising both A and B. “Or” should, however, be construed to exclude those aspects presented that cannot be combined without contradiction (e.g., a composition pH that is between 9 and 10 or between 7 and 8).

As used herein, the term “comprise” is used in its non-limiting sense to mean that items following the word are included, but items not specifically mentioned are not excluded.

As used herein, the term “Cannabis” refers to the genus of flowering plants in the family Cannabaceae regardless of species, subspecies, or subspecies variety classification. At present, there is no general consensus whether plants of genus Cannabis are comprised of a single or multiple species (McPartland & Guy, 2017). For example some describe Cannabis plants as a single species, C. sativa L., with multiple subspecies (Small & Cronquist, 1976)(McPartland & Small, 2020) while others classify Cannabis plants into multiple species, most commonly as C. sativa L. and C. indica Lam. and sometimes additionally as C. ruderalis Janisch. (Schultes et al., 1974), depending on multiple criteria including morphology, geographic origin, chemical content, and genetic measurements. Regardless, all plants of genus Cannabis can interbreed and produce fertile offspring (Small, 1972)

The term “strain” as used herein refers to different varieties of the plant genus Cannabis. For example, the term “strain” can refer to different pure or hybrid varieties of Cannabis plants. In some instances, the Cannabis strain of the present technology can by a hybrid of two strains. Different Cannabis strains often exhibit distinct chemical compositions with characteristic levels of cannabinoids and terpenes, as well as other components. Differing cannabinoid and terpene profiles associated with different Cannabis strains can be useful for the treatment of different diseases, or for treating different subjects with the same disease.

As used herein, the term “cannabinoid” refers to a chemical compound belonging to a class of secondary compounds commonly found in plants of genus Cannabis, but also encompasses synthetic and semi-synthetic cannabinoids and any enantiomers thereof. In an embodiment, the cannabinoid is a compound found in a plant, e.g., a plant of genus Cannabis, and is sometimes referred to as a phytocannabinoid. In one embodiment, the cannabinoid is a compound found in a mammal, sometimes called an endocannabinoid. In one embodiment, the cannabinoid is made in a laboratory setting, sometimes called a synthetic cannabinoid. In one embodiment, the cannabinoid is derived or obtained from a natural source (e.g. plant) but is subsequently modified or derivatized in one or more different ways in a laboratory setting, sometimes called a semi-synthetic cannabinoid.

Synthetic cannabinoids and semi-synthetic cannabinoids encompass a variety of distinct chemical classes, for example and without limitation: the classical cannabinoids structurally related to THC, the non-classical cannabinoids (cannabimimetics) including the aminoalkylindoles, 1,5 diarylpyrazoles, quinolines, and arylsulfonamides as well as eicosanoids related to endocannabinoids.

In another embodiment, a cannabinoid is one of a class of diverse chemical compounds that may act on cannabinoid receptors such as CB₁ and CB₂ in cells that alter neurotransmitter release in the brain.

In many cases, a cannabinoid can be identified because its chemical name will include the text string “*cannabi*”. However, there are a number of cannabinoids that do not use this nomenclature, such as for example those described herein.

As used herein, the expression “% by weight” is calculated based on dry weight of the total material.

As used herein, “THC content”, “CBD content” and “CBG content” may include “THCA content”, “CBDA content” and “CBGA content”, respectively.

Within the context of this disclosure, where reference is made to a particular cannabinoid, each of the acid and/or decarboxylated forms are contemplated as both single molecules and mixtures. In addition, salts of cannabinoids are also encompassed, such as salts of cannabinoid carboxylic acids. As well, any and all isomeric, enantiomeric, or optically active derivatives are also encompassed. In particular, where appropriate, reference to a particular cannabinoid includes both the “A Form” and the “B Form”. For example, it is known that THCA has two isomers, THCA-A in which the carboxylic acid group is in the 1 position between the hydroxyl group and the carbon chain (A Form) and THCA-B in which the carboxylic acid group is in the 3 position following the carbon chain (B Form).

In some embodiments of the present disclosure, the cannabinoid is a cannabinoid dimer. The cannabinoid may be a dimer of the same cannabinoid (e.g. THC-THC) or different cannabinoids. In an embodiment of the present disclosure, the cannabinoid may be a dimer of THC, including for example Cannabisol.

In an embodiment, a cannabinoid may occur in its free form, or in the form of a salt; an acid addition salt of an ester; an amide; an enantiomer; an isomer; a tautomer; a prodrug; a derivative of an active agent of the present invention; different isomeric forms (for example, enantiomers and diastereoisomers), both in pure form and in admixture, including racemic mixtures; enol forms.

As used herein, the expressions “nucleic acid,” “nucleic acid molecule,” “oligonucleotide,” and “polynucleotide” are each used herein to refer to a polymer of at least three nucleotides. In some embodiments, a nucleic acid comprises deoxyribonucleic acid (DNA). In some embodiments comprises ribonucleic acid (RNA). In some embodiments, a nucleic acid is single stranded. In some embodiments, a nucleic acid is double stranded. In some embodiments, a nucleic acid comprises both single and double stranded portions. Unless otherwise stated, the terms encompass nucleic acid-like structures with synthetic backbones, as well as amplification products. In some embodiments, nucleic acids of the present disclosure are linear nucleic acids.

As used herein, the term “gene” refers to a part of the genome that code for a product (e.g., an RNA product and/or a polypeptide product). A “gene sequence” is a sequence that includes at least a portion of a gene (e.g., all or part of a gene) and/or regulatory elements associated with a gene. In some embodiments, a gene includes coding sequence; in some embodiments, a gene includes non-coding sequence. In some particular embodiments, a gene may include both coding (e.g., exonic) and non-coding (e.g., intronic) sequences. In some embodiments, a gene may include one or more regulatory elements (e.g., a promoter) that, for example, may control or impact one or more aspects of gene expression (e.g., cell-type-specific expression, inducible expression, etc.).

As used herein, the expression “coding sequence” refers to a sequence of a nucleic acid or its complement, or a part thereof, that: i) can be transcribed to an mRNA sequence that can be translated to produce a polypeptide or a fragment thereof; or ii) an mRNA sequence that can be translated to produce a polypeptide or a fragment thereof. Coding sequences include exons in genomic DNA or immature primary RNA transcripts, which are joined together by the cell's biochemical machinery to provide a mature mRNA.

As used herein, the term “mutation” refers to a change introduced into a parental sequence, including, but not limited to, substitutions, insertions, deletions (including truncations). The consequences of a mutation include, but are not limited to, the creation of a new character, property, function, phenotype or trait not found in the protein encoded by the parental sequence, or the increase or reduction/elimination of an existing character, property, function, phenotype or trait not found in the protein encoded by the parental sequence.

The expression “degree or percentage of sequence homology” refers herein to the degree or percentage of sequence identity between two sequences after optimal alignment. Percentage of sequence identity (or degree of identity) is determined by comparing two aligned sequences over a comparison window, where the portion of the peptide or polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity.

As used herein, the term “isolated” refers to nucleic acids or polypeptides that have been separated from their native environment, including but not limited to virus, proteins, glycoproteins, peptide derivatives or fragments or polynucleotides. For example, the expression “isolated nucleic acid molecule” as used herein refers to a nucleic acid substantially free of cellular material or culture medium when produced by recombinant DNA techniques, or chemical precursors, or other chemicals when chemically synthesized. An isolated nucleic acid is also substantially free of sequences, which naturally flank the nucleic acid (i.e. sequences located at the 5′ and 3′ ends of the nucleic acid) from which the nucleic acid is derived.

Two nucleotide sequences or amino-acids are said to be “identical” if the sequence of nucleotide residues or amino-acids in the two sequences is the same when aligned for maximum correspondence as described below. Sequence comparisons between two (or more) peptides or polynucleotides are typically performed by comparing sequences of two optimally aligned sequences over a segment or “comparison window” to identify and compare local regions of sequence similarity. Optimal alignment of sequences for comparison may be conducted by the local homology algorithm of Smith and Waterman, Ad. App. Math 2: 482 (1981), by the homology alignment algorithm of Needleman and Wunsch, J. Mol. Biol. 48: 443 (1970), by the search for similarity method of Pearson and Lipman, Proc. Natl. Acad. Sci. (U.S.A.) 85: 2444 (1988), by computerized implementation of these algorithms (GAP, BESTFIT, FASTA, and TFASTA in the Wisconsin Genetics Software Package, Genetics Computer Group (GCG), 575 Science Dr., Madison, Wis.), or by visual inspection. Other alignment programs may also be used such as: “Multiple sequence alignment with hierarchical clustering”, F. CORPET, 1988, Nucl. Acids Res., 16 (22), 10881-10890.

As used herein, the expression “conservative substitutions” refers to a substitution made in an amino acid sequence of a polypeptide without disrupting the structure or function of the polypeptide. Conservative amino acid substitutions may be accomplished by substituting amino acids with similar hydrophobicity, polarity, and R-chain length for one another. Additionally, by comparing aligned sequences of homologous proteins from different species, conservative amino acid substitutions may be identified by locating amino acid residues that have been mutated between species without altering the basic functions of the encoded proteins. Amino acid substitutions that are conservative are typically as follows: i) hydrophilic: Alanine (Ala) (A), Proline (Pro) (P), Glycine (Gly) (G), Glutamic acid (Glu) (E), Aspartic acid (Asp) (D), Glutamine (Gin) (Q), Asparagine (Asn) (N), Serine (Ser) (S), Threonine (Thr) (T); ii) Sulphydryl: Cysteine (Cys) (C); iii) Aliphatic: Valine (Val) (V), Isoleucine (Ile) (I), Leucine (Leu) (L), Methionine (Met) (M); iv) Basic: Lysine (Lys) (K), Arginine (Arg) (R), Histidine (His) (H); and v) Aromatic: Phenylalanine (Phe) (F), Tyrosine (Tyr) (Y), Tryptophan (Trp) (W).

An “expression system” as used herein refers to reagents and components (e.g. in a kit) and/or solutions comprising said reagents and components for recombinant protein expression, wherein the expression system is cell free and includes optionally translation competent extracts of whole cells and/or other translation machinery reagents or components optionally in a solution, said reagents and components optionally including RNA polymerase, one or more regulatory protein factors, one or more transcription factors, ribosomes, and tRNA, optionally supplemented with cofactors and nucleotides, and the specific gene template of interest. Chemical based expression systems are also included, optionally using unnaturally occurring amino acids. In some instances, the expression systems of the present technology are in vitro expression system.

The expressions “transformed with”, “transfected with”, “transformation” and “transfection” are intended to encompass introduction of nucleic acid (e.g. a construct) into a cell by one of many possible techniques known in the art.

The term “primer” as used herein, typically refers to oligonucleotides that hybridize in a sequence specific manner to a complementary nucleic acid molecule (e.g., a nucleic acid molecule comprising a target sequence). In some embodiments, a primer will comprise a region of nucleotide sequence that hybridizes to at least 8, e.g., at least 10, at least 15, at least 20, at least 25, or 20 to 60 nucleotides of a target nucleic acid (i.e., will hybridize to a sequence of the target nucleic acid). In general, a primer sequence is identified as being either “complementary” (i.e., complementary to the coding or sense strand (+)), or “reverse complementary” (i.e., complementary to the anti-sense strand (−)). In some embodiments, the term “primer” may refer to an oligonucleotide that acts as a point of initiation of a template-directed synthesis using methods such as PCR (polymerase chain reaction) under appropriate conditions (e.g., in the presence of four different nucleotide triphosphates and a polymerization agent, such as DNA polymerase in an appropriate buffer solution containing any necessary reagents and at suitable temperature(s)). Such a template directed synthesis is also called “primer extension.” For example, a primer pair may be designed to amplify a region of DNA using PCR. Such a pair will include a “forward primer” and a “reverse primer” that hybridize to complementary strands of a DNA molecule and that delimit a region to be synthesized and/or amplified.

As used herein, the expression “wild-type” refers to a typical or common form existing in nature; in some embodiments it is the most common form.

The term “antibody” as used herein is intended to include monoclonal antibodies, polyclonal antibodies, and chimeric antibodies. The antibody may be from recombinant sources and/or produced in transgenic animals. Antibody binding fragment: The term “antibody binding fragment” as used herein is intended to include Fab, Fab′, F(ab′)2, scFv, dsFv, ds-scFv, dimers, minibodies, diabodies, and multimers thereof and bispecific antibody fragments. Antibodies can be fragmented using conventional techniques. Antibodies may be monospecific, bispecific, trispecific or of greater multispecificity. Multispecific antibodies may immunospecifically bind to different epitopes of a cannabichromenic acid synthase and/or or a solid support material. Antibodies may be prepared using methods known to those skilled in the art. Isolated native or recombinant polypeptides may be utilized to prepare antibodies. See, for example, Kohler et al. (1975) Nature 256:495-497; Kozbor et al. (1985) J. Immunol. Methods, 81:31-42; Cote et al. (1983) Proc Natl Acad Sci., 80:2026-2030; and Cole et al. (1984) Mol Cell Biol., 62:109-120, for the preparation of monoclonal antibodies; Huse et al. (1989) Science, 246:1275-1281, for the preparation of monoclonal Fab fragments; and, Pound (1998) Immunochemical Protocols, Humana Press, Totowa, N.J., for the preparation of phagemid or B-lymphocyte immunoglobulin libraries to identify antibodies.

As used herein, “allele” generally refers to an alternative nucleic acid sequence at a particular locus; the length of an allele can be as small as 1 nucleotide base but is typically larger. For example, a first allele can occur on one chromosome, while a second allele occurs on a second homologous chromosome, e.g., as occurs for different chromosomes of a heterozygous individual, or between different homozygous or heterozygous individuals in a population. A “favorable allele” is the allele at a particular locus that confers, or contributes to, an agronomically desirable phenotype, or alternatively, is an allele that allows the identification of susceptible plants that can be removed from a breeding program or planting. A favorable allele of a marker is a marker allele that segregates with the favorable phenotype, or alternatively, segregates with susceptible plant phenotype, therefore providing the benefit of identifying disease prone plants. A favorable allelic form of a chromosome interval is a chromosome interval that includes a nucleotide sequence that contributes to superior agronomic performance at one or more genetic loci physically located on the chromosome interval.

“Allele frequency” refers to the frequency (proportion or percentage) at which an allele is present at a locus within an individual, within a line, or within a population of lines. For example, for an allele “A”, diploid individuals of genotype “AA”, “Aa”, or “aa” have allele frequencies of 1.0, 0.5, or 0.0, respectively. One can estimate the allele frequency within a line by averaging the allele frequencies of a sample of individuals from that line. Similarly, one can calculate the allele frequency within a population of lines by averaging the allele frequencies of lines that make up the population. For a population with a finite number of individuals or lines, an allele frequency can be expressed as a count of individuals or lines (or any other specified grouping) containing the allele. An allele positively correlates with a trait when it is linked to it and when presence of the allele is an indicator that the desired trait or trait form will occur in a plant comprising the allele. An allele negatively correlates with a trait when it is linked to it and when presence of the allele is an indicator that a desired trait or trait form will not occur in a plant comprising the allele.

“Crossed” or “cross” means to produce progeny via fertilization (e.g. cells, seeds or plants) and includes crosses between plants (sexual) and self fertilization (selfing).

“Gene” refers to a heritable sequence of DNA, i.e., a genomic sequence, with functional significance. The term “gene” can also be used to refer to, e.g., a cDNA and/or a mRNA encoded by a genomic sequence, as well as to that genomic sequence.

“Genotype” is the genetic constitution of an individual (or group of individuals) at one or more genetic loci, as contrasted with the observable trait (the phenotype). Genotype is defined by the allele(s) of one or more known loci that the individual has inherited from its parents. The term genotype can be used to refer to an individual's genetic constitution at a single locus, at multiple loci, or, more generally, the term genotype can be used to refer to an individual's genetic make-up for all the genes in its genome. A “haplotype” is the genotype of an individual at a plurality of genetic loci. Typically, the genetic loci described by a haplotype are physically and genetically linked, i.e., on the same chromosome interval. The terms “phenotype,” or “phenotypic trait” or “trait” refers to one or more trait of an organism, i.e., the detectable characteristics of a cell or organism which can be influenced by genotype. The phenotype can be observable to the naked eye, or by any other means of evaluation known in the art, e.g., microscopy, biochemical analysis, genomic analysis, an assay for a particular disease resistance, etc. In some cases, a phenotype is directly controlled by a single gene or genetic locus, i.e., a “single gene trait”. In other cases, a phenotype is the result of several genes.

“Germplasm” refers to genetic material of or from an individual (e.g., a plant), a group of individuals (e.g., a plant line, variety or family), or a clone derived from a line, variety, species, or culture. The germplasm can be part of an organism or cell or can be separate from the organism or cell. In general, germplasm provides genetic material with a specific molecular makeup that provides a physical foundation for some or all of the hereditary qualities of an organism or cell culture. As used herein, germplasm includes cells, seed or tissues from which new plants may be grown, or plant parts, such as leaves, stems, pollen, or cells that can be cultured into a whole plant.

“Polymorphism” means the presence of one or more variations in a population. A polymorphism may manifest as a variation in the nucleotide sequence of a nucleic acid or as a variation in the amino acid sequence of a protein. Polymorphisms include the presence of one or more variations of a nucleic acid sequence or nucleic acid feature at one or more loci in a population of one or more individuals. The variation may comprise but is not limited to one or more nucleotide base changes, the insertion of one or more nucleotides or the deletion of one or more nucleotides. A polymorphism may arise from random processes in nucleic acid replication, through mutagenesis, as a result of mobile genomic elements, from copy number variation and during the process of meiosis, such as unequal crossing over, genome duplication and chromosome breaks and fusions. The variation can be commonly found or may exist at low frequency within a population, the former having greater utility in general plant breeding and the latter may be associated with rare but important phenotypic variation. Useful polymorphisms may include single nucleotide polymorphisms (SNPs), insertions or deletions in DNA sequence (Indels), simple sequence repeats of DNA sequence (SSRs), a restriction fragment length polymorphism, and a tag SNP. A genetic marker, a gene, a DNA-derived sequence, a RNA-derived sequence, a promoter, a 5′ untranslated region of a gene, a 3′ untranslated region of a gene, microRNA, siRNA, a tolerance locus, a satellite marker, a transgene, mRNA, ds mRNA, a transcriptional profile, and a methylation pattern may also comprise polymorphisms. In addition, the presence, absence, or variation in copy number of the preceding may comprise polymorphisms.

As used herein, “marker”, “genetic marker”, “molecular marker”, “marker nucleic acid”, and “marker locus” refer to a nucleotide sequence or encoded product thereof (e.g., a protein) used as a point of reference when identifying a linked locus. A marker can be derived from genomic nucleotide sequence or from expressed nucleotide sequences (e.g., from a spliced RNA, a cDNA, etc.), or from an encoded polypeptide, and can be represented by one or more particular variant sequences, or by a consensus sequence. In another sense, a marker is an isolated variant or consensus of such a sequence. The term also refers to nucleic acid sequences complementary to or flanking the marker sequences, such as nucleic acids used as probes or primer pairs capable of amplifying the marker sequence. A “marker probe” is a nucleic acid sequence or molecule that can be used to identify the presence of a marker locus, e.g., a nucleic acid probe that is complementary to a marker locus sequence. Alternatively, in some aspects, a marker probe refers to a probe of any type that is able to distinguish (i.e., genotype) the particular allele that is present at a marker locus. A “marker locus” is a locus that can be used to track the presence of a second linked locus, e.g., a linked locus that encodes or contributes to expression of a phenotypic trait. For example, a marker locus can be used to monitor segregation of alleles at a locus, such as a QTL, that are genetically or physically linked to the marker locus. Thus, a “marker allele,” alternatively an “allele of a marker locus” is one of a plurality of polymorphic nucleotide sequences found at a marker locus in a population that is polymorphic for the marker locus.

As used herein, “marker” also refers to nucleic acid sequences complementary to the genomic sequences, such as nucleic acids used as probes. Markers corresponding to genetic polymorphisms between members of a population can be detected by methods well-established in the art. These include, for example and without limitation, PCR-based sequence specific amplification methods, detection of restriction fragment length polymorphisms (RFLP), detection of isozyme markers, detection of polynucleotide polymorphisms by allele specific hybridization (ASH), detection of amplified variable sequences of the plant genome, detection of self-sustained sequence replication, detection of simple sequence repeats (SSRs), detection of single nucleotide polymorphisms (SNPs), or detection of amplified fragment length polymorphisms (AFLPs). Well established methods are also known for the detection of expressed sequence tags (ESTs) and SSR markers derived from EST sequences and randomly amplified polymorphic DNA (RAPD).

A “population of plants” or “plant population” means a set comprising any number, generally more than one, of individuals, objects, or data from which samples are taken for evaluation, e.g. estimating QTL effects. Most commonly, the terms relate to a breeding population of plants from which members are selected and crossed to produce progeny in a breeding program. A population of plants can include the progeny of a single breeding cross or a plurality of breeding crosses and can be either actual plants or plant derived material, or in silica representations of the plants. The population members need not be identical to the population members selected for use in subsequent cycles of analyses or those ultimately selected to obtain final progeny plants. Often, a plant population is derived from a single biparental cross but may also derive from two or more crosses between the same or different parents. Although a population of plants may comprise any number of individuals, those of skill in the art will recognize that plant breeders commonly use population sizes ranging from one or two hundred individuals to several thousand, and that the highest performing 5-20% of a population is what is commonly selected to be used in subsequent crosses in order to improve the performance of subsequent generations of the population.

“Recombinant” in reference to a nucleic acid or polypeptide indicates that the material (e.g., a recombinant nucleic acid, gene, polynucleotide, polypeptide, etc.) has been altered by human intervention. The term recombinant can also refer to an organism that harbors recombinant material, e.g., a plant that comprises a recombinant nucleic acid is considered a recombinant plant.

As used herein, the expression “plant part” refers to any part of a plant including but not limited to the embryo, shoot, root, stem, seed, stipule, leaf, petal, flower bud, flower, ovule, bract, trichome, branch, petiole, internode, bark, pubescence, tiller, rhizome, frond, blade, ovule, pollen, stamen, and the like. The two main parts of plants grown in some sort of media, such as soil or vermiculite, are often referred to as the “above-ground” part, also often referred to as the “shoots”, and the “below-ground” part, also often referred to as the “roots.” Plant part may also include certain extracts such as kief or hash which includes Cannabis trichomes or glands.

As used herein, the term “chemotype” refers to the cannabinoid chemical phenotype in individual Cannabis strains. In general, chemotype is primarily determined by, but not limited to, chemical ratios or predominance of CBD, THC, and CBG and/or their acid counterparts CBDA, THCA, and CBGA present in mature or semi-mature Cannabis flower. For example, Small and Beckstead assigned chemotypes based on ratios of THCA and CBDA: plants producing primarily THCA (Type I), CBDA (Type III) or both THCA and CBDA (Type II) (Small & Beckstead, 1973). Much rarer CBGA-dominant hemp plants were later identified as a new chemotype (Type IV) (Fournier et al., 1987).

Based on thorough genetic inheritance studies, de Meijer et al. postulated that most Cannabis strains contain a co-dominant cannabinoid synthase locus (B) that contains either THCAS (Bt) or CBDAS (Bd) (de Meijer et al., 2003). They hypothesized if functional versions of these enzymes are present they will convert CBGA into THCAS or CBDAS, respectively. Since Cannabis is diploid, plants homozygous for THCAS (Bt/Bt, Type I) will be dominant for THCA, plants homozygous for CBDAS (Bd/Bd, Type III) will be dominant for CBDA, and heterozygous plants (Bt/Bd, Type II) will produce significant amounts of both. They also proposed that recessive, minimally-functional or non-functional versions of CBDAS or THCAS could exist, which they termed Bo, and that homozygous Bo/Bo alleles would prevent conversion of CBGA to CBDA or THCA leading to CBGA accumulation and CBGA-dominant (Type IV) plants.

Whole-genome DNA sequencing on crosses of THC-dominant drug type Cannabis and CBD-dominant hemp showed THCAS and CBDAS alleles reside at different, highly linked loci on the same chromosome and are inherited as co-dominant alleles, fitting the Bt/Bd allele segregation hypothesis (Laverty et al., 2019)(Grassa et al., 2018). DNA sequencing studies often showed the presence of multiple CBDAS and/or THCAS sequences in individual Cannabis strains, with many sequences being incomplete, containing early stop codons or not being expressed at the mRNA level (Weiblen et al., 2015)(Onofri et al., 2015). It was further shown that THCAS and CBDAS alleles contained neighboring duplicated THCAS or CBDAS genes in a “gene cluster” but that a single, full-length copy of THCAS or CBDAS was present in these gene clusters, presumably mediating chemotype via the Bt/Bd allele hypothesis (Grassa et al., 2018).

Two groups isolated putative minimally-functional or non-functional CBDAS mutant enzymes (Bo) isolated from European or Russian hemp cultivars and bred CBGA-dominant hemp lines with hypothesized Bo/Bo genotypes (de Meijer & Hammond, 2005, Pacifico et al., 2006).

The expression “minimally-functional”, as used herein refers to a mutant CBDAS or THCAS allele that when homozygous and present as the only full-length coding sequence for the cannabinoid synthase at the B locus prevents conversion of the majority of CBGA to the terminal acid cannabinoid but retains a minimal catalytic activity such that more than about 5% of total cannabinoid fraction is the corresponding terminal cannabinoid (e.g. CBDA/CBD for CBDAS and THCA/THC for THCAS).

The expression “non-functional”, as used herein refers to a mutant CBDAS or THCAS allele that when homozygous and present as the only full-length coding sequence for the cannabinoid synthase at the B locus prevents conversion of the majority of CBGA to the terminal acid cannabinoid and does not retain catalytic activity such that less than about 5% of total cannabinoid fraction is the corresponding terminal cannabinoid (e.g. CBDA/CBD for CBDAS and THCA/THC for THCAS).

To date, sequencing of THCAS and CBDAS nucleic acids from CBGA-dominant lines has shown three unique Bo alleles caused by single nucleotide polymorphisms (SNPs): THCAS G706C, CBDAS C1426T and CBDAS G1465A (Onofri et al., 2015). These cause amino acid changes THCAS E236Q, CBDAS P476S and CBDAS G489R, respectively that produce minimally-functional or non functional enzymes.

CBDAS G1465A SNP is the only reported non-functional mutant which produced only trace amounts of CBDA and THCA while accumulating greater than about 99% of the cannabinoid fraction as CBGA. THCAS G706C SNP accumulated around 11% of the cannabinoid fraction as THCA and CBDAS C1426T SNP accumulated around 6% of the cannabinoid fraction as CBDA indicating these are minimally-functional alleles.

Prior to the current invention the only commercialized CBGA-dominant cannabis strains described are derived from European or Russian fiber hemp cultivars with minimally-functional or nonfunctional CBDAS alleles. Thus there is a need for more genetic diversity of CBG-dominant Cannabis strains, especially those with the Bo allele derived from a THCAS mutant background, for three major reasons.

First, CBGA-dominant Cannabis bred via a minimally-functional or non-functional THCAS allele is expected to be genetically distinct from CBGA-dominant Cannabis bred via a minimally-functional or non-functional CBDAS allele because both THCAS and CBDAS are located in a region of approximately million base pairs of minimally-recombining DNA on the same chromosome (Laverty et al., 2019).

Other genes found within this region that have evolutionarily diverged between THCAS and CBDAS lineages may be responsible for other important traits and are expected to be inherited as linked genes. As such, genetic admixture may be impossible or difficult for genes and traits linked on this chromosome. For example, other metabolites produced by the Cannabis plant are posited as being responsible for the “entourage effect”, which is a therapeutic benefit observed from Cannabis cannabinoid extracts containing other metabolites but not observed from isolated single cannabinoids (Russo, 2011). If genes for these important other metabolites are located in this linkage group, it is expected to be beneficial to start with breeding material closer to medical cannabis (e.g. THCAS allele) than hemp (e.g. CBDAS allele)

Second, Cannabis with less than 0.3% total THC by dry weight is recognized as hemp, whereas Cannabis with greater than 0.3% THC is recognized as drug-type and is heavily regulated. Development of CBGA-dominant strains from a non-functional, but not a minimally-functional THCAS genetic background will allow high-cannabinoid producing plants to be cultivated as hemp and not as drug-type cannabis. For example the only CBGA-dominant strain to be reported derived from a mutant THCAS allele (THCAS G706C, (Onofri et al., 2015)) was derived from a minimally-functional THCAS that produced around 11% cannabinoid fraction as THCA/THC and would therefore test over the legal limit of 0.3% THC at around 3% total cannabinoids which is significantly less than the high cannabinoid strains used today.

Third, high cannabinoid producing strains (with cannabinoids greater than about 10% by dry weight) dominate the market for both THC and CBD production. Described existing CBGA-dominant cannabis strains derived from CBDAS alleles from European or Russian hemp are bred for fiber production and not cannabinoid isolation. Fiber hemp routinely produces less than about 2% cannabinoids by dry weight and increasing cannabinoid levels requires interbreeding with drug strains, often THC-dominant, which are often illegal or highly regulated depending on jurisdiction. Thus the current invention provides CBGA-dominant Cannabis from a high-cannabinoid producing background which has more immediate value and requires less breeding to stabilize.

The nucleic acid sequence of wild type THCA synthase is provided in SEQ ID NO: 1:
ATGAATTGCTCAGCATTTTCCTTTTGGTTTGTTTGCAAAATAATATTTTTCTTTCTCTCATTCCATATCCAAATTTCA
ATAGCTAATCCTCGAGAAAACTTCCTTAAATGCTTCTCAAAACATATTCCCAACAATGTAGCAAATCCAAAACTCG
TATACACTCAACACGACCAATTGTATATGTCTATCCTGAATTCGACAATACAAAATCTTAGATTCATCTCTGATACA
ACCCCAAAACCACTCGTTATTGTCACTCCTTCAAATAACTCCCATATCCAAGCAACTATTTTATGCTCTAAGAAAGT
TGGCTTGCAGATTCGAACTCGAAGCGGTGGCCATGATGCTGAGGGTATGTCCTACATATCTCAAGTCCCATTTGTT
GTAGTAGACTTGAGAAACATGCATTCGATCAAAATAGATGTTCATAGCCAAACTGCGTGGGTTGAAGCCGGAGCT
ACCCTTGGAGAAGTTTATTATTGGATCAATGAGAAGAATGAGAATCTTAGTTTTCCTGGTGGGTATTGCCCTACTGT
TGGCGTAGGTGGACACTTTAGTGGAGGAGGCTATGGAGCATTGATGCGAAATTATGGCCTTGCGGCTGATAATATT
ATTGATGCACACTTAGTCAATGTTGATGGAAAAGTTCTAGATCGAAAATCCATGGGAGAAGATCTGTTTTGGGCTA
TACGTGGTGGTGGAGGAGAAAACTTTGGAATCATTGCAGCATGGAAAATCAAACTGGTTGCTGTCCCATCAAAGT
CTACTATATTCAGTGTTAAAAAGAACATGGAGATACATGGGCTTGTCAAGTTATTTAACAAATGGCAAAATATTGC
TTACAAGTATGACAAAGATTTAGTACTCATGACTCACTTCATAACAAAGAATATTACAGATAATCATGGGAAGAAT
AAGACTACAGTACATGGTTACTTCTCTTCAATTTTTCATGGTGGAGTGGATAGTCTAGTCGACTTGATGAACAAGA
GCTTTCGTGAGTTGGGTATTAAAAAAACTGATTGCAAAGAATTTAGCTGGATTGATACAACCATCTTCTACAGTGG
TGTTGTAAATTTTAACACTGCTAATTTTAAAAAGGAAATTTTGCTTGATAGATCAGCTGGGAAGAAGACGGCTTTC
TCAATTAAGTTAGACTATGTTAAGAAACCAATTCCAGAAACTGCAATGGTCAAAATTTTGGAAAAATTATATGAAG
AAGATGTAGGAGCTGGGATGTATGTGTTGTACCCTTACGGTGGTATAATGGAGGAGATTTCAGAATCAGCAATTCC
ATTCCCTCATCGAGCTGGAATAATGTATGAACTTTGGTACACTGCTTCCTGGGAGAAGCAAGAAGATAATGAAAA
CCATATAAACTGGGTTCGAAGTGTTTATAATTTTACGACTCCTTATGTGTCCCAAAATCCAAGATTGGCGTATCTCA
ATTATAGGGACCTTGATTTAGGAAAAACTAATCATGCGAGTCCTAATAATTACACACAAGCACGTATTTGGGGTGA
AAAGTATTTTGGTAAAAATTTTAACAGGTTAGTTAAGGTGAAAACTAAAGTTGATCCCAATAATTTTTTTAGAAAC
GAACAAAGTATCCCACCTCTTCCACCGCATCATCATTAA
The amino acid sequence encoded by the nucleic acid sequence of wild type THCA
synthase is provided in SEQ ID NO: 3:
MNCSAFSFWFVCKIIFFFLSFHIQISIANPRENFLKCFSKHIPNNVANPKLVYTQHDQLYMSILNSTIQNLRFISDTTPKPLVI
VTPSNNSHIQATILCSKKVGLQIRTRSGGHDAEGMSYISQVPFVVVDLRNMHSIKIDVHSQTAWVEAGATLGEVYYWIN
EKNENLSFPGGYCPTVGVGGHFSGGGYGALMRNYGLAADNIIDAHLVNVDGKVLDRKSMGEDLFWAIRGGGGENFGII
AAWKIKLVAVPSKSTIFSVKKNMEIHGLVKLFNKWQNIAYKYDKDLVLMTHFITKNITDNHGKNKTTVHGYFSSIFHGG
VDSLVDLMNKSFRELGIKKTDCKEFSWIDTTIFYSGVVNFNTANFKKEILLDRSAGKKTAFSIKLDYVKKPIPETAMVKIL
EKLYEEDVGAGMYVLYPYGGIMEEISESAIPFPHRAGIMYELWYTASWEKQEDNEKHINWVRSVYNFTTPYVSQNPRL
AYLNYRDLDLGKTNHASPNNYTQARIWGEKYFGKNFNRLVKVKTKVDPNNFFRNEQSIPPLPPHHH

The present technology stems from the recognition that certain genotypes are associated with altered cannabinoid synthesis or an altered cannabinoid profile. In some embodiments, the present technology recognizes that certain variants (e.g., mutated) of tetrahydrocannabinolic acid synthase (THCAS) gene sequences can provide insight regarding accumulation of cannabigerolic acid (CBGA) and/or cannabigerol (CBG) in a Cannabis plant.

In some embodiments, the present technology relates to a minimally functional THCAS allele. In some implementations of these embodiments, the minimally functional THCAS allele causes a decrease of production of the associated gene product or causes the associated gene product not to function properly as compared to the wild type associated gene product.

In some implementations of these embodiments, the minimally functional THCAS allele comprises an activity-altering change that causes a decrease of production of the associated gene product or causes the associated gene product not to function properly as compared to the wild type associated gene product.

In some implementations of these embodiments, the activity-altering change of the minimally functional THCAS allele comprises a SNP in the region that encodes for amino acids spanning between position 330 and 360 of the corresponding polypeptide.

In some further implementations the minimally functional THCAS allele comprises a SNP at position 1064. In some instances, the SNP is G1064A.

In some embodiments, the present technology relates to a non-functional THCA synthase allele. In some implementations of these embodiments, the non-functional THCAS allele causes a lack of production of the associated gene product or causes the associated gene product not to function properly as compared to the wild type associated gene product.

In some implementations of these embodiments, the non-functional THCAS allele comprises an activity-altering change that causes a lack of production of the associated gene product or causes the associated gene product not to function properly as compared to the wild type associated gene product.

In some implementations of these embodiments, the activity-altering change of the non functional THCAS allele comprises a SNP in the region that encodes for amino acids spanning between position 330 and 360 of the corresponding polypeptide.

In some further implementations the non-functional THCAS allele comprises a SNP at position 1064. In some instances, the SNP is G1064A.

In some instances, the minimally functional and/or the non-functional THCAS allele impedes or prevents conversion of CBGA to THCA leading to a plant (e.g., Cannabis plant) with a CBGA-dominant cannabinoid fraction and/or a CBG-dominant cannabinoid fraction.

In some embodiments, the present technology relates to a nucleic acid molecule having the nucleic acid sequence as depicted in SEQ ID NO: 2:

ATGAATTGCTCAGCATTTTCCTTTTGGTTTGTTTGCAAAATAATATTTTTCTTTCTCTCaTTCCATATCCAAATTTCAA
TAGCTAATCCTCGAGAAAACTTCCTTAAATGCTTCTCAAAACATATTCCCAACAATGTAGCAAATCCAAAACTCGT
ATACACTCAACACGACCAATTGTATATGTCTATCCTGAATTCGACAATACAAAATCTTAGATTCATCTCTGATACA
ACCCCAAAACCACTCGTTATTGTCACTCCTTCAAATAACTCCCATATCCAAGCAACTATTTTATGCTCTAAGAAAGT
TGGCTTGCAGATTCGAACTCGAAGCGGTGGCCATGATGCTGAGGGTATGTCCTACATATCTCAAGTCCCATTTGTT
GTAGTAGACTTGAGAAACATGCATTCGATCAAAATAGATGTTCATAGCCAAACTGCGTGGGTTGAAGCCGGAGCT
ACCCTTGGAGAAGTTTATTATTGGATCAATGAGAAGAATGAGAATCTTAGTTTTCCTGGTGGGTATTGCCCTACTGT
TGGCGTAGGTGGACACTTTAGTGGAGGAGGCTATGGAGCATTGATGCGAAATTATGGCCTTGCGGCTGATAATATT
ATTGATGCACACTTAGTCAATGTTGATGGAAAAGTTCTAGATCGAAAATCCATGGGAGAAGATCTGTTTTGGGCTA
TACGTGGTGGTGGAGGAGAAAACTTTGGAATCATTGCAGCATGGAAAATCAAACTGGTTGCTGTCCCATCAAAGT
CTACTATATTCAGTGTTAAAAAGAACATGGAGATACATGGGCTTGTCAAGTTATTTAACAAATGGCAAAATATTGC
TTACAAGTATGACAAAGATTTAGTACTCATGACTCACTTCATAACAAAGAATATTACAGATAATCATGGGAAGAAT
AAGACTACAGTACATGGTTACTTCTCTTCAATTTTTCATGGTGGAGTGGATAGTCTAGTCGACTTGATGAACAAGA
GCTTTCGTGAGTTGGGTATTAAAAAAACTGATTGCAAAGAATTTAGCTGGATTGATACAACCATCTTCTACAATGG
TGTTGTAAATTTTAACACTGCTAATTTTAAAAAGGAAATTTTGCTTGATAGATCAGCTGGGAAGAAGACGGCTTTC
TCAATTAAGTTAGACTATGTTAAGAAACCAATTCCAGAAACTGCAATGGTCAAAATTTTGGAAAAATTATATGAAG
AAGATGTAGGAGCTGGGATGTATGTGTTGTACCCTTACGGTGGTATAATGGAGGAGATTTCAGAATCAGCAATTCC
ATTCCCTCATCGAGCTGGAATAATGTATGAACTTTGGTACACTGCTTCCTGGGAGAAGCAAGAAGATAATGAAAA
GCATATAAACTGGGTTCGAAGTGTTTATAATTTTACGACTCCTTATGTGTCCCAAAATCCAAGATTGGCGTATCTCA
ATTATAGGGACCTTGATTTAGGAAAAACTAATCATGCGAGTCCTAATAATTACACACAAGCACGTATTTGGGGTGA
AAAGTATTTTGGTAAAAATTTTAACAGGTTAGTTAAGGTGAAAACTAAAGTTGATCCCAATAATTTTTTTAGAAAC
GAACAAAGTATCCCACCTCTTCCACCGCATCATCATTAA

In some embodiments, the present technology relates to an isolated nucleic acid molecule having at least about 75%, or at least about 80%, or at least about 85%, at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity to SEQ ID NO: 2, while conserving the activity-altering change of the present disclosure that causes in a minimally functional or non-functional THCAS of the present technology.

In some embodiments, the present technology relates to nucleic acid molecules that hybridize to the above disclosed sequences. Hybridization conditions may be stringent in that hybridization will occur if there is at least about a 96% or about a 97% sequence identity with the nucleic acid molecule in SEQ ID NO: 2. The stringent conditions may include those used for known Southern hybridizations such as, for example, incubation overnight at 42° C. in a solution having 50% formamide, 5×SSC (150 mM NaCl, 15 mM trisodium citrate), 50 mM sodium phosphate (pH 7.6), 5×Denhardt's solution, 10% dextran sulfate, and 20 mg/mL denatured, sheared salmon sperm DNA, following by washing the hybridization support in 0.1×SSC at about 65° C. Other known hybridization conditions are well known and are described in Sambrook et al., Molecular Cloning: A Laboratory Manual, Third Edition, Cold Spring Harbor, N.Y. (2001).

In some embodiments, the isolated nucleic acid of the present technology comprises a nucleic acid sequence as set forth in SEQ ID NO: 2 or a fragment thereof, wherein the fragment therefor conserves the activity-altering change of the present disclosure that gives rise to a minimally functional or to a non functional THCAS of the present disclosure.

Fragments contemplated by the present technology, but not limited to, include fragments having a nucleic acid sequence as set forth in SEQ ID NO: 5, 6, 7, 8, 9, 10, 11 and 12 as well as sequences with at least or about 85% or more sequence identity thereto are also contemplated.

In some embodiments, the isolated nucleic acid molecule of the present technology comprises at least and/or up to or about 15, at least and/or up to or about 20 at least and/or up to or about 25, at least and/or up to or about 30, at least and/or up to or about 40 at least and/or up to or about 50, at least and/or up to or about 60, at least and/or up to or about 70, at least and/or up to or about 80, at least and/or up to or about 90, at least and/or up to 100, at least or up to or about 200, at least or up to or about 300, at least or up or about 400, at least or up to or about 500, at least or up to or about 600, at least or up to or about 700, at least or up to or about 800, at least or up to or about 900, at least or up to or about 1000, at least or up to or about 1100, at least or up to or about 1200, at least or up to or about 1300, at least or up to or about 1400 or at least or up to or about 1500 or about 1600 contiguous nucleotides of SEQ ID NO: 2. For example, the nucleic acid molecule can be from 15 contiguous nucleotides up to 1500 contiguous nucleotides or any range or number of nucleotides there between.

The length of the nucleic acid molecule described above will depend on the intended use. For example, if the intended use is as a primer or probe, for example, for PCR amplification or for screening a library, the length of the nucleic acid molecule will be less than the full length sequence, such as a fragment of for example, about 15 to about 50 nucleotides, or at least about 15 nucleotides of SEQ ID NO: 2 and/or its complement. In these embodiments, the primers or probes may be substantially identical to a highly conserved region of the nucleic acid sequence or may be substantially identical to either the 5′ or 3′ end of the DNA sequence. In some cases, these primers or probes may use universal bases in some positions so as to be ‘substantially identical’ but still provide flexibility in sequence recognition. Suitable primer and probe hybridization conditions are well known in the art.

“Amplification” or “amplifying” in the context of nucleic acid amplification, is any process whereby additional copies of a selected nucleic acid (or a transcribed form thereof) are produced. In some embodiments, an amplification based marker technology is used wherein a primer or amplification primer pair is admixed with genomic nucleic acid isolated from the first plant or germplasm, and wherein the primer or primer pair is complementary or partially complementary to at least a portion of the locus, and is capable of initiating DNA polymerization by a DNA polymerase using the plant genomic nucleic acid as a template. The primer or primer pair is extended in a DNA polymerization reaction having a DNA polymerase and a template genomic nucleic acid to generate at least one amplicon. In other embodiments, plant RNA is the template for the amplification reaction.

In an embodiment, the nucleic acid molecule can be used as a primer and for example comprises the nucleic acid sequence as set forth in any one of SEQ ID NO: 5, 6, 7, 8, 9, 10, 11 or 12.

In general, synthetic methods for making oligonucleotides, including probes, primers, molecular beacons, PNAs (peptide nucleic acids), LNAs (locked nucleic acids), etc., are known. For example, oligonucleotides can be synthesized chemically according to a solid phase phosphoramidite triester method. Oligonucleotides, including modified oligonucleotides, can also be ordered from a variety of commercial sources. Nucleic acid probes to the marker loci can be cloned and/or synthesized. Any suitable label can be used with a probe of the disclosure. Detectable labels suitable for use with nucleic acid probes include, for example, any composition detectable by spectroscopic, radioisotopic, photochemical, biochemical, immunochemical, electrical, optical or chemical means. Useful labels include biotin for staining with labeled streptavidin conjugate, magnetic beads, fluorescent dyes, radio labels, enzymes, and colorimetric labels. Other labels include ligands which bind to antibodies labeled with fluorophores, chemiluminescent agents, and enzymes. A probe can also constitute radio labeled PCR primers that are used to generate a radio labeled amplicon. It is not intended that the nucleic acid probes of the disclosure be limited to any particular size. In some embodiments, the primers of the disclosure are radiolabelled, or labeled by any suitable means (e.g., using a non-radioactive fluorescent tag), to allow for rapid visualization of the different size amplicons following an amplification reaction without any additional labeling step or visualization step. In some embodiments, the primers are not labeled, and the amplicons are visualized following their size resolution, e.g., following agarose gel electrophoresis. In some embodiments, ethidium bromide staining of the PCR amplicons following size resolution allows visualization of the different size amplicons. It is not intended that the primers of the disclosure be limited to generating an amplicon of any particular size. For example, the primers used to amplify the marker loci and alleles herein are not limited to amplifying the entire region of the relevant locus.

In an embodiment, the nucleic acid is conjugated to and/or comprises a heterologous moiety, such as a unique tail, purification tag or detectable label. The unique tail can be a specific nucleic acid sequence. The nucleic acid can for example be end labelled (5′ or 3′) or the label can be incorporated randomly during synthesis.

In one embodiment, the present technology provides an isolated nucleic acid that encodes for the polypeptide having an amino acid sequence as set forth in SEQ ID NO: 4 or a fragment thereof, wherein SEQ ID NO: 4 is as follows:

MNCSAFSFWFVCKIIFFFLSFHIQISIANPRENFLKCFSKHIPNNVANPKLVYTQHDQLYMSILNSTIQNLRFISDTTPKPLVI
VTPSNNSHIQATILCSKKVGLQIRTRSGGHDAEGMSYISQVPFVVVDLRNMHSIKIDVHSQTAWVEAGATLGEVYYWIN
EKNENLSFPGGYCPTVGVGGHFSGGGYGALMRNYGLAADNIIDAHLVNVDGKVLDRKSMGEDLFWAIRGGGGENFGI
IAAWKIKLVAVPSKSTIFSVKKNMEIHGLVKLFNKWQNIAYKYDKDLVLMTHFITKNITDNHGKNKTTVHGYFSSIFHG
GVDSLVDLMNKSFRELGIKKTDCKEFSWIDTTIFYNGVVNFNTANFKKEILLDRSAGKKTAFSIKLDYVKKPIPETAMVK
ILEKLYEEDVGAGMYVLYPYGGIMEEISESAIPFPHRAGIMYELWYTASWEKQEDNEKHINWVRSVYNFTTPYVSQNPR
LAYLNYRDLDLGKTNHASPNNYTQARIWGEKYFGKNFNRLVKVKTKVDPNNFFRNEQSIPPLPPHHH

In some implementations, the isolated nucleic acid is deleted of all or part of the nucleotides that encode the signal sequence, for example nucleotides 5-27 of SEQ ID NO: 1 or nucleotides 5-27 of SEQ ID NO: 2.

In some embodiments, the present technology relates to an isolated polypeptide having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98% or at least about 99% identity to the amino acid sequence as set forth in SEQ ID NO: 4. In some implementations of these embodiments, the isolated polypeptide comprises the activity-altering change that results in a minimally functional and/or a non functional THCAS that impedes conversion of CBGA to THCA.

In some embodiments, the present technology relates to an antibody that specifically binds a polypeptide as set forth in SEQ ID NO: 4 or to fragments thereof. In some instances, the antibody is a purified antibody. By “purified” is meant that a given antibody or fragment thereof, whether one that has been removed from nature (isolated from blood serum) or synthesized (produced by recombinant means), has been increased in purity, wherein “purity” is a relative term, not “absolute purity”. In particular aspects, a purified antibody is 60% free, preferably at least about 75% free, and more preferably at least about 90% free from other components with which it is naturally associated or associated following synthesis.

In some embodiments, the present technology relates a construct or an in vitro expression system having an isolated nucleic acid molecule having at least, greater than or about 75% sequence identity to SEQ ID NO: 2. Accordingly, the present technology further relates to a method for preparing a construct or in vitro expression system including such a sequence, or a fragment thereof, for introduction of the sequence or partial sequence in a sense or anti-sense orientation, or a complement thereof, into a cell.

In some embodiments, an extract of the recombinant organism described herein or of a part thereof, such as a recombinant plant extract, comprises an increased level of CBGA and/or CBG. Accordingly, an aspect of these embodiments includes a cannabinoid or a composition comprising CBGA and/or CBG, produced according to a method or system described herein.

Is some embodiments, the present technology relates to a recombinant organism, host cell or germ tissue (e.g. seed) of the organism comprising a nucleic acid molecule having at least 15 contiguous nucleotides of SEQ ID NO: 2 and/or a construct comprising said isolated and/or purified nucleic acid molecule. In some instances of these embodiments, the at least 15 contiguous nucleotides of SEQ ID NO: 2 include the activity-altering change that results in a minimally functional or non-functional THCAS impeding conversion of CBGA to THCA.

In an embodiment, the recombinant organism, cell and/or germ tissue expresses a polypeptide having at least and/or up to about 150, about 175, about 200, about 225, or about 250 amino acids of the polypeptide sequence and optionally at least about 90% sequence identity to as set forth in SEQ ID NO: 4, which conserving the activity-altering change that results in a minimally functional or non functional THCAS impeding conversion of CBGA to THCA.

The recombinant expression vectors of the present technology may also contain nucleic acid sequences which encode a heterologous polypeptide (e.g. fusion moiety) producing a fusion polypeptide when a nucleic acid of interest encoding a polypeptide is introduced into the vector in frame. The heterologous polypeptide can provide for increased expression of the recombinant protein; increased solubility of the recombinant protein; and/or aid in the purification of the target recombinant protein by acting as a ligand in affinity purification. For example, a proteolytic cleavage site may be added between the target recombinant protein to allow separation of the recombinant protein from the fusion moiety subsequent to purification of the fusion polypeptide. Typical fusion expression vectors include pGEX (Amrad Corp., Melbourne, Australia), pMal (New England Biolabs, Beverly, Mass.) and pRIT5 (Pharmacia, Piscataway, N.J.) which fuse glutathione S-transferase (GST), maltose E binding protein, or protein A, respectively, to the recombinant protein.

Preferably, the recombinant organism is a recombinant plant, recombinant multicellular microorganism or recombinant insect. Plants are preferably of the genus Cannabis. Microorganisms are preferably bacteria (e.g. Escherichia coli) or yeast (e.g. Saccharomyces cerevisiae, Pichia pastoris). Microorganisms that are unicellular can be considered organisms or cells, including host cells. Insect is preferably Spodoptera frugiperda.

In some embodiments, the present technology also provides for organisms, tissues or cells such as Cannabis plants, Cannabis tissue and Cannabis cells having minimally functional or a non functional THCAS that impedes conversion of CBGA to THCA.

In some embodiments, the present technology also provides for organisms, tissues or cells such as Cannabis plants, Cannabis tissue and Cannabis cells having minimally functional or a non-functional THCAS that causes an increase in CBGA and/or CBG in the organisms, tissues or cells.

In some embodiments, the present technology also provides for organisms, tissues or cells that comprise the nucleic acids and/or the polypeptides as defined herein. In some embodiments, the organisms, tissues or cells are plants, plant tissues or plant cells that exhibit THCAS activity. In some instances, such plants are Cannabis plants and such plant tissues and plant cells are Cannabis tissue and Cannabis cells.

Plants, and in particular Cannabis plants, containing the THCAS nucleotide sequences of the present technology may be created via known plant transformation methods for example Agrobacterium-mediated transformation, transformation via particle bombardment, pollen tube or protoplast transformation. In these methodological approaches, the gene of interest is incorporated into the genome of the target organism. For example, tissue culture and Agrobacterium mediated transformation of hemp is described in Feeney and Punja, 2003.

Recombinant expression vectors can be introduced into host cells to produce a transformed host cell. Prokaryotic and/or eukaryotic cells can be transformed with nucleic acid by, for example, electroporation or calcium chloride-mediated transformation. For example, nucleic acid can be introduced into mammalian cells via conventional techniques such as calcium phosphate or calcium chloride co-precipitation, DEAE-dextran mediated transfection, lipofectin, viral mediated methods, electroporation or microinjection. Suitable methods for transforming and transfecting cells can be found in Sambrook et al. (Molecular Cloning: A Laboratory Manual, 3rd Edition, Cold Spring Harbor Laboratory Press, 2001), and other laboratory textbooks.

Suitable host cells include a wide variety of eukaryotic cells and prokaryotic cells. For example, the nucleic acids and proteins of the disclosure may be expressed in plant cells, yeast cells or mammalian cells. Plant cells are preferably of the genus Cannabis. Microorganisms are preferably bacteria (e.g. Escherichia coli) or yeast (e.g. Saccharomyces cerevisiae, Pichia pastoris).

Other suitable host cells can be found in Goeddel, Gene Expression Technology: Methods in Enzymology 185, Academic Press, San Diego, Calif. (1991). In addition, the proteins of the disclosure may be expressed in prokaryotic cells, such as Escherichia coli (Zhang et al., Science 303(5656): 371-3 (2004)). In addition, a Pseudomonas-based expression system such as Pseudomonas fluorescens can be used (US Patent Application Publication No. US 2005/0186666).

In some embodiments, the present technology also relates to recombinant cells comprising a nucleic acid molecule or polynucleotide of the disclosure. In an embodiment, the nucleic acid molecule results in an increased level of CBGA and/or CBG in the recombinant cell.

Recombinant organisms, cells and tissues described herein may have altered levels of cannabinoid compounds and in particular may have altered levels of CBGA and/or CBG. Expression of the nucleic acid and amino acids sequences of the present technology will result in expression of a minimally functional or non-functional THCAS enzyme which may result in decreased conversion of CBGA to THCA and in turn may result in an increased production of CBGA and/or CBG.

In some embodiments, the Cannabis plants, cells and tissues described herein have a CBG content ranging between about 1% and about 30% by weight; or between about 1% and about 25% by weight; or between about 1% and about 20% by weight; or between about 1% and about 15% by weight; or between about 1% and about 10% by weight; or between about 5% and about 30% by weight; or between about 5% and about 25% by weight; or between about 5% and about 20% by weight; or between about 5% and about 15% by weight; or between about 5% and about 10% by weight. In some implementations of these embodiments, the CBG content includes CBGA content.

In some embodiments, the Cannabis plants, cells and tissues described herein have a CBG content of at least about 1% by weight; or at least about 5% by weight; or at least about 10% by weight; or at least about 15% by weight; or at least about 20% by weight; or at least about 25% by weight; or at least about 30% by weight. In some implementations of these embodiments, the CBG content includes CBGA content.

In some embodiments, the Cannabis plants, cells and tissues described herein have a CBGA content ranging between about 1% and about 30% by weight; or between about 1% and about 25% by weight; or between about 1% and about 20% by weight; or between about 1% and about 15% by weight; or between about 1% and about 10% by weight; or between about 5% and about 30% by weight; or between about 5% and about 25% by weight; or between about 5% and about 20% by weight; or between about 5% and about 15% by weight; or between about 5% and about 10% by weight.

In some embodiments, the Cannabis plants, cells and tissues described herein have a CBGA content of at least about 1% by weight; or at least about 5% by weight; or at least about 10% by weight; or at least about 15% by weight; or at least about 20% by weight; or at least about 25% by weight; or at least about 30% by weight.

Expression of the activity-altered THCAS enzyme of the present disclosure compared to a control which has normal levels of the THCAS enzyme activity grown under similar or identical conditions will result in increased levels of CBGA and/or CBG, for example, about 1-about 50%, about 2-about 50%, about 5-about 50%, about 10-about 50%, about 25-about 50%, or about 1-about 25% (w/w). In some instances, the control is of the same variety.

Expression of the activity-altered THCAS enzyme of the present disclosure compared to a control which has normal levels of the THCAS enzyme activity for the same variety grown under similar or identical conditions will result in increased levels of CBGA and/or CBG, by at least about 1.5 time, at least about 2 times, at least about 5 times, at least about 10 times, at least about 11 times, at least about 12 times, at least about 13 times, at least about 14 times, at least about 15 times, at least about 20 times, at least about 25 times, at least about 50 times, or at least about 75 times, or about 100 times, or about 250 times, or about 500 times or about 1000 times, or about 2000 times, or greater than about 2000 times.

Expression of the activity-altered THCAS enzyme of the present disclosure compared to a control which has normal levels of the THCAS enzyme activity for the same variety grown under similar or identical conditions will result in CBGA/THCA ratio of at least about 10:1, at least about 25:1, at least about 50:1, at least about 75:1, at least about 100:1, at least about 150:1, at least about 200:1, at least about 250:1, at least about 300:1, at least about 350:1, at least about 400:1, at least about 450:1, or at least about 500:1.

Expression of the activity-altered THCAS enzyme of the present disclosure compared to a control which has normal levels of the THCAS enzyme activity for the same variety grown under similar or identical conditions will result in CBG/THC ratio of at least about 10:1, at least about 25:1, at least about 50:1, at least about 75:1, at least about 100:1, at least about 150:1, at least about 200:1, at least about 250:1, at least about 300:1, at least about 350:1, at least about 400:1, at least about 450:1, or at least about 500:1.

Expression of the activity-altered THCAS enzyme of the present disclosure compared to a control which has normal levels of the THCAS enzyme activity for the same variety grown under similar or identical conditions will result in CBGA/CBDA ratio of at least about 10:1, at least about 25:1, at least about 50:1, at least about 75:1, at least about 100:1, at least about 150:1, at least about 200:1, at least about 250:1, at least about 300:1, at least about 350:1, at least about 400:1, at least about 450:1, or at least about 500:1.

Expression of the activity-altered THCAS enzyme of the present disclosure compared to a control which has normal levels of the THCAS enzyme activity for the same variety grown under similar or identical conditions will result in CBG/CBD ratio of at least about 10:1, at least about 25:1, at least about 50:1, at least about 75:1, at least about 100:1, at least about 150:1, at least about 200:1, at least about 250:1, at least about 300:1, at least about 350:1, at least about 400:1, at least about 450:1, or at least about 500:1.

In some embodiments, the Cannabis plants, cells and tissues described herein have a CBG content ranging between about 50% and about 99% by weight of the total cannabinoid content; or between about 75% and about 99% by weight of the total cannabinoid content; or between about 85% and about 99% by weight of the total cannabinoid content; or between about 50% and about 95% by weight of the total cannabinoid content; or between about 75% and about 95% by weight of the total cannabinoid content; or between about 85% and about 95% by weight of the total cannabinoid content. In some implementations of these embodiments, the CBG content includes CBGA content.

In some embodiments, the Cannabis plants, cells and tissues described herein have a CBG content of at least about 50% by weight of the total cannabinoid content; or at least about 75% by weight of the total cannabinoid content; or at least about 85% by weight of the total cannabinoid content; or at least about 90% by weight of the total cannabinoid content; or at least about 95% by weight of the total cannabinoid content. In some implementations of these embodiments, the CBG content includes CBGA content.

In some embodiments, the Cannabis plants, cells and tissues described herein have a CBGA content ranging between about 50% and about 99% by weight of the total cannabinoid content; or between about 75% and about 99% by weight of the total cannabinoid content; or between about 85% and about 99% by weight of the total cannabinoid content; or between about 50% and about 95% by weight of the total cannabinoid content; or between about 75% and about 95% by weight of the total cannabinoid content; or between about 85% and about 95% by weight of the total cannabinoid content.

In some embodiments, the Cannabis plants, cells and tissues described herein have a CBGA content of at least about 50% by weight of the total cannabinoid content; or at least about 75% by weight of the total cannabinoid content; or at least about 85% by weight of the total cannabinoid content; or at least about 90% by weight of the total cannabinoid content; or at least about 95% by weight of the total cannabinoid content.

In some embodiments, the Cannabis plants, cells and tissues described herein have a THC content of between about 0.01% and about 0.5% by weight, or between about 0.05% and about 0.2% by weight, or between about 0.05% and about 0.10% by weight. In some instances, the THC content includes THCA content.

In some embodiments, the Cannabis plants, cells and tissues described herein have a CBD content of between about 0.01% and about 0.20% by weight, or between about 0.01% and about 0.10% by weight. In some instances, the CBD content includes CBDA content.

In some instances, the control is of the same variety.

In Cannabis plants the transmission of the activity-altering change defined herein and the enhanced production of CBGA and/or CBG could be achieved through breeding and selection as well as genetic engineering with the use of genes encoding the enzymes of cannabinoid biosynthetic pathways, e.g. the THCAS gene in this disclosure.

In some embodiments, the present technology relates to methods of altering levels of CBGA and/or CBG compounds in an organism, cell or tissue, said method comprising using a nucleic acid molecule of the present disclosure or a fragment thereof to cause an activity-altered THCAS to be expressed in the organism, cell or tissue. In some implementations of these embodiments, the levels of CBGA and/or CBG compounds is increased by making the recombinant cells expressing the minimally-functional or the non-functional THCAS of the present technology.

In one embodiment, the present technology relates to methods for increasing the production of CBGA and/or CBG in cells of an organism. In some instances, the organism produces THCAS (e.g., a Cannabis plant). In some implementations of this embodiment, the method comprises introducing into the cells of the organism, a vector comprising a nucleic acid comprising SEQ ID NO: 2 or a fragment thereof conserving the activity-altering change as disclosed herein. The vector comprises a nucleic acid having at least or about 75% sequence identity to SEQ ID NO: 2 while retaining the activity-altering change to produce recombinant cells. The method may further comprise the step of culturing and/or growing the recombinant cells under conditions that permit expression of the nucleic acid; and optionally isolating and/or purifying CBGA and/or CBG. The recombinant cell can be transiently expressing, inducibly expressing and/or stably expressing.

In some embodiments, a Cannabis plant genome to be modified by the methods of the present technology includes a THCA synthase gene sequence. In some instances, a Cannabis plant genome to be modified by the methods of the present technology includes a wild-type THCA synthase gene sequence. In some instances, the Cannabis plant genome is homozygous for a wild-type THCA synthase gene sequence or is heterozygous for a wild-type THCA synthase gene sequence or is homozygous for a variant THCA synthase gene sequence. In some embodiments, a Cannabis plant genome is heterozygous for a variant THCA synthase gene sequence. In some embodiments, a Cannabis plant genome includes a THCA synthase gene sequence that is or comprises a sequence that is 70%, 75%, 80%, 85%, 85%, 87%, 88%, 89%, 90%, 91%, 92%, 93%, 94%, 95%, 96%, 97%, 98%, 99%, or 100% idenmical to SEQ ID NO: 1, or a portion thereof.

The recombinant expression vector of the present technology, in addition to containing a nucleic acid molecule or polynucleotide disclosed herein, may contain regulatory sequences for the transcription and translation of the inserted nucleic acid molecule. Suitable regulatory sequences may be derived from a variety of sources, including bacterial, fungal, viral, mammalian, or insect genes (For example, see the regulatory sequences described in Goeddel, Gene Expression Technology: Methods in Enzymology 185, Academic Press, San Diego, Calif. (1990)). Selection of appropriate regulatory sequences is dependent on the host cell chosen as discussed below, and may be readily accomplished by one of ordinary skill in the art. Examples of such regulatory sequences include: a transcriptional promoter and enhancer or RNA polymerase binding sequence, a ribosomal binding sequence, including a translation initiation signal. Additionally, depending on the host cell chosen and the vector employed, other sequences, such as an origin of replication, additional DNA restriction sites, enhancers, and sequences conferring inducibility of transcription may be incorporated into the expression vector. As will also be apparent to persons skilled in the art, and as described elsewhere (Meyer, 1995; Datla et al., 1997), it is possible to utilize promoters to direct any intended up- or down-regulation of transgene expression using constitutive promoters (e.g., those based on CaMV35S), or by using promoters which can target gene expression to particular cells, tissues (e.g., napin promoter for expression of transgenes in developing seed cotyledons), organs (e.g., roots, leaves), to a particular developmental stage, or in response to a particular external stimulus (e.g., heat shock).

In some embodiments, the present technology relates to a method for detecting the presence of a THCAS gene sequence or a portion thereof that comprises the activity-altering change of the present disclosure. The method includes amplifying a THCAS gene sequence or portion thereof comprising the activity-altering change from a sample that comprises nucleic acid from a Cannabis plant. The amplification of a THCAS gene sequence includes contacting nucleic acid from a Cannabis plant with a forward THCAS primer that is complementary to a sequence that is 200-1000 nucleotides upstream of a THCAS open reading frame. In some embodiments, a forward THCAS primer is complementary to a sequence that is 200 to 800 nucleotides, 200 to 600 nucleotides, 200 to 400 nucleotides upstream of a THCAS open reading frame. In some certain embodiments, a forward THCAS primer is complementary to a sequence that is at least 70%, 75%, 80%, 85%, 85%, 87%, 88%, 89%, 90%, 91%, 92%, 93%, 94%, 95%, 96%, 97%, 98%, 99%, or 100% identical to a portion of SEQ ID NO: 2. In some certain embodiments, a forward THCAS primer is at 20 to 60 nucleotides long. The amplification of a THCAS gene sequence includes contacting nucleic acid from a Cannabis plant with a reverse THCAS primer that is complementary to a sequence that is 200-1000 nucleotides downstream of a THCAS open reading frame. In some embodiments, a reverse THCAS primer is complementary to a sequence that is 200 to 800 nucleotides, 200 to 600 nucleotides, 200 to 400 nucleotides downstream of a THCAS open reading frame. In some certain embodiments, a reverse THCAS primer is complementary to a sequence that is at least 70%, 75%, 80%, 85%, 85%, 87%, 88%, 89%, 90%, 91%, 92%, 93%, 94%, 95%, 96%, 97%, 98%, 99%, or 100% identical to a portion of SEQ ID NO: 2. In some certain embodiments, a reverse THCAS primer is about 20 to 60 nucleotides long.

In some embodiments, a method of the present disclosure includes detecting the presence of a polymorphism within a THCAS gene sequence in a sample that comprises nucleic acid from the Cannabis plant. In some embodiments, a polymorphism within a THCAS gene sequence results in a polypeptide that comprises an amino acid change between position 330 and 360 of the amino acid sequence.

In some embodiments, the present technology relates to a method for controlling the conversion of CBGA to THCA in Cannabis. In some implementations, the method comprises obtaining an endonuclease enzyme which targets a nucleic acid sequence coding for THCAS and introducing the endonuclease enzyme into the genome of a plant of genus Cannabis. In some implementations of these embodiments, the endonuclease enzyme is made in vitro. The introduction of the endonuclease enzyme may be accomplished through inoculating the plant with a bacteria comprising a genetic sequence for an endonuclease enzyme. Once inoculated, the bacteria make plant cells which will then produce the endonuclease enzyme. In some instances, inoculating comprises placing the Cannabis plant in a vacuum chamber with a bacterial solution comprising the endonuclease enzyme and removing air drawing the bacterial solution comprising the endonuclease enzyme into the plan. In some instances, the inoculation comprises spraying the Cannabis plant with an endonuclease enzyme. In some instances, spraying is accomplished using biolistic particles bombardment.

In some implementations of these embodiments, the endonuclease enzyme is a CRISPR/Cas9 system. As used herein, the term “CRISPR” refers to an acronym that means Clustered Regularly Interspaced Short Palindromic Repeats of DNA sequences. CRISPR is a series of repeated DNA sequences with unique DNA sequences in between the repeats. RNA transcribed from the unique strands of DNA serves as guides for directing cleaving. CRISPR is used as a gene editing tool. In one embodiment, CRISPR is used in conjunction with a Cas9 protein. As used herein, the term “Cas” refers to CRISPR associated proteins that act as enzymes cutting the genome at specific sequences. Cas9 refers to a specific group of proteins known in the art. RNA sequences made from CRISPR direct Cas9 enzymes to cut certain sequences found in the genome. Other classes of Cas are also acceptable. In some instances, the CRISPR/Cas9 system cleaves one or two chromosomal strands at known Cas9 protein domains. In one embodiment, one of the two chromosomal strands is mutated. In one embodiment, two of the two chromosomal strands are mutated. As used herein, the term “chromosomal strand” refers to a sequence of DNA within the chromosome. When the CRISPR/Cas9 system cleaves the chromosomal strands, the strands are cut leaving the possibility of one or two strands being mutated, either the template strand or coding strand. The CRISPR/Cas9 system cleaves both strands inducing non-homologous end joining (NHEJ) and then an insertion of a DNA sequence that includes the activity-altering change thereby causing the encoded protein to mutate and become minimally functional or non-functional. In one embodiment, the CRISPR/Cas9 system cleaves both strands causing homology directed repair (HDR) to occur. In some instances, a donor DNA strand is inserted into the space between the cleaved strands preventing random mutation. In one embodiment, the donor DNA strand is a DNA sequence coding for a minimally functional or non-functional THCAS enzyme.

In some embodiments, the activity-altering change results in a THCAS enzyme that is minimally functional or non-functional.

In one embodiment, the methods disclosed herein comprise a RNA guide. As used herein, the term “RNA guide” refers to a strand of RNA recognizing a specific sequence of genetic material and directing where the endonuclease enzyme to cut. In one embodiment, the RNA guide directs the endonuclease enzyme to cleave chromosomal strands coding for a cannabinoid synthesis enzyme. In one embodiment, the RNA guide directs the CRISPR/Cas9 system to cleave chromosomal strands coding for a cannabinoids synthesis enzyme. In one embodiment, the RNA guide directs the CRISPR/Cas9 system to target a THCAS expression gene. Within the context of this disclosure, other examples of endonuclease enzymes include SpCas9 from Streptococcus pyrogenes and others. Additionally, SpCas9 have differing Protospacer Adjacent Motif (PAM) sequences from NGG, which may offer other advantages. In one example, a SpCas9 has a smaller coding sequence. Other examples of proteins that work with CRISPRs or RNA guides include Cpf1, which can be used for cutting DNA strands with overhanging ends instead of blunt ends, or C2c2 for cutting RNA with an RNA guide. As used herein, the term “PAM” refers to a short DNA base pair sequence immediately following the DNA sequence targeted by an endonuclease enzyme.

In one embodiment, the methods disclosed herein comprise an endonuclease enzyme and an RNA guide. In one embodiment, the methods disclosed herein comprise a guide RNA transcribed in vitro. In one embodiment, the methods disclosed herein comprise a guide RNA transcribed in vivo.

In one embodiment, the methods disclosed herein comprise introducing a Cas9 enzyme and guide RNA expression cassette into the genome.

In one embodiment, the CRISPR/Cas9 system cleaves a sequence of a functional THCA synthase expression gene. Within the context of this disclosure, cleaving a sequence of a functional gene causes a sequence change changing the functionality of the THCAS enzyme expressed from the gene. In some embodiments, the present disclosure provides kits comprising materials useful for amplification and detection and/or sequencing of Cannabis plant nucleic acid (e.g., DNA). In some embodiments, Cannabis plant nucleic acid sample includes detection of all or part of a THCAS gene sequence as described herein. In some embodiments, a kit in accordance of the present disclosure is portable.

Suitable amplification reaction reagents that can be included in an inventive kit include, for example, one or more of: buffers; enzymes having polymerase activity; enzyme cofactors such as magnesium or manganese; salts; nicotinamide adenide dinuclease (NAD); and deoxynucleoside triphosphates (dNTPs) such as, for example, deoxyadenosine triphospate; deoxyguanosine triphosphate, deoxycytidine triphosphate and deoxythymidine triphosphate, biotinylated dNTPs, suitable for carrying out the amplification reactions.

In some embodiments, a kit comprises 1, 2, 3, 4, 5, 6, 7, 8, 9, 10, 11, 12, 13, 14, 15, 16, 17, 18, 19, 20, or more primer sequences for in vitro nucleic acid amplification. Primer sequences may be suitable for in vitro nucleic acid amplification with any of the methods described herein (e.g., QT-PCR, LAMP, etc.). In some embodiments, a kit of the present disclosure includes reagents suitable to perform a colorimetric LAMP assay for amplification of one or more Cannabis gene sequences as described herein.

Depending on the procedure, a kit may further include one or more of: wash buffers and/or reagents, hybridization buffers and/or reagents, labeling buffers and/or reagents, and detection means. The buffers and/or reagents included in a kit are preferably optimized for the particular amplification/detection technique for which a kit is intended. Protocols for using these buffers and reagents for performing different steps of the procedure may also be included in a kit.

In some embodiments, a kit may further include one or more reagents for preparation of nucleic acid from a plant sample. For example, a kit may further include one or more of a lysis buffer, a DNA preparation solution (e.g., a solution for extraction and/or purification of DNA). Kits may also contain reagents for the isolation of nucleic acids from biological specimen prior to amplification. Protocols for using these reagents for performing different steps of the procedure may also be included in a kit.

Furthermore, kits may be provided with an internal control as a check on the amplification procedure and to prevent occurrence of false negative test results due to failures in the amplification procedure. An optimal control sequence is selected in such a way that it will not compete with the target nucleic acid sequence in the amplification reaction (as described above).

In some embodiments, a kit may further include reagents for an amplification assay to characterize the gender of a Cannabis plant.

Reagents may be supplied in a solid (e.g., lyophilized) or liquid form. Kits of the present disclosure may optionally comprise different containers (e.g., vial, ampoule, test tube, flask or bottle) for each individual buffer and/or reagent. In some embodiments, each component will generally be suitable as aliquoted in its respective container or provided in a concentrated form. Other containers suitable for conducting certain steps of inventive amplification/detection assay(s) may also be provided. Individual containers of a kit are preferably maintained in close confinement for commercial sale.

A kit may also comprise instructions for using the amplification reaction reagents, primer sets, primer/probe sets according to the present disclosure. Instructions for using a kit according to one or more methods of the present disclosure may comprise instructions for processing the biological sample, extracting nucleic acid molecules, and/or performing one or more amplification reactions; and/or instructions for interpreting results.

### EXAMPLES

The examples below are given so as to illustrate the practice of various embodiments of the present disclosure. They are not intended to limit or define the entire scope of this disclosure. It should be appreciated that the disclosure is not limited to the particular embodiments described and illustrated herein but includes all modifications and variations falling within the scope of the disclosure as defined in the appended embodiments.

### Example 1—Identification of Activity-Altering SNP in THCA Synthase of CBG-Dominant Cannabis Plant

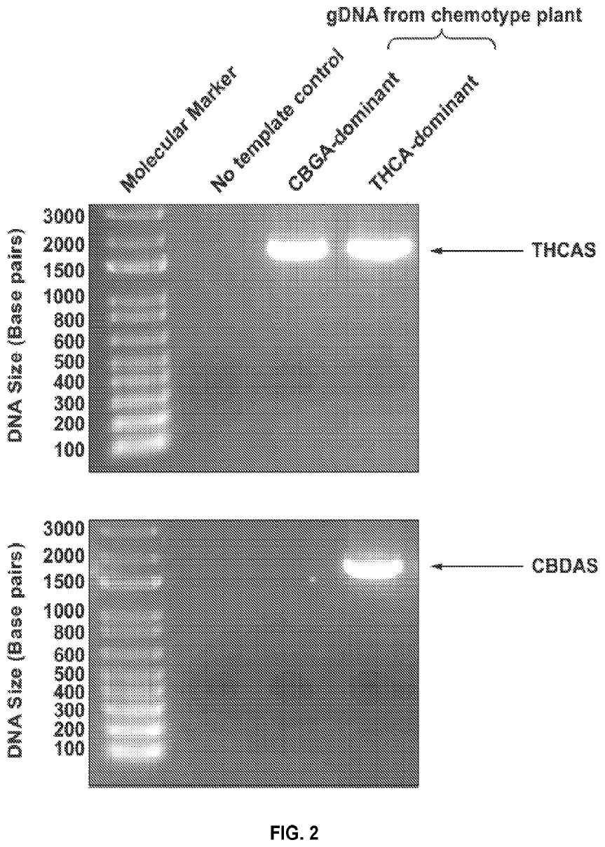

To assess potential THCAS and CBDAS alleles in a CBGA-dominant Cannabis strain and a THCA-dominant medicinal cannabis strain Balmoral®, primer sets specific for THCAS and CBDAS were designed on unique consensus sequences in genomic regions 5′ and 3′ to the THCAS and CBDAS coding regions. Young leaf tissue was collected (20 mg) from a CBGA-dominant Cannabis strain and a THCA-dominant Balmoral® strain and was homogenized by bead beating. Genomic DNA was extracted using a Maxwell® RSC Plant DNA Kit and Maxwell® RSC instrument (Promega®) using manufacturer's instructions. A PCR was performed on genomic DNA template using Q5 enzyme (New England Biolabs®) and for THCAS primers having nucleic acid sequences: forward: 5′-TTTATCACATGTGACTATTTAATGAC-3′ (SEQ ID NO: 5) and reverse: 5′-TGATTAACTAATTGATAAGGGAAATATATC-3′ (SEQ ID NO: 6) or for CBDAS primers having nucleic acid sequences: forward: 5′-CCCTGCTCCAATATATAAAGC-3′ (SEQ ID NO: 7) and reverse: 5′-ATACACAGTACATCCGGAC-3′ (SEQ ID NO: 8) using manufacturer's instructions with the following thermal cycler program: 1) 98° C. for 30 sec; 2) 98° C. for 10 sec; 3) 59° C. for 20 sec for THCAS or 61° C. for 20 sec for CBDAS; 4) 72° C. for 1 min (repeat 2-4 34X); 5) 72° C. for 2 min; and 6) Hold at 4° C. PCR product was separated on a 1% agarose gel and visualized with SYBR™ Safe DNA gel stain (ThermoFisher®). A control PCR was used without template DNA. Results showed a CBGA-dominant strain only amplified for THCAS whereas Balmoral® amplified for both THCAS and CBDAS (FIG. 2). Because Balmoral® is a THCA-dominant strain with little to no CBDA and CBD detected (Table 1) it is assumed that the CBDAS allele(s) amplified is/are non-functional because CBDAS alleles with frameshift mutations and/or early stop codons are prevalent in some THCA-dominant medicinal Cannabis strains (Weiblen et al., 2015; Laverty et al., 2019).

To assess the THCAS allele from a CBGA-dominant Cannabis and THCA-dominant Balmoral®, the ˜1700 bp bands for THCAS in FIG. 2 were excised and DNA was purified using Zymoclean™ Gel DNA recovery kit (Zymo Research®). The extracted DNAs were subjected to Sanger sequencing using primers having nucleic acid sequences as depicted in SEQ ID NO: 5 and SEQ ID NO: 6.

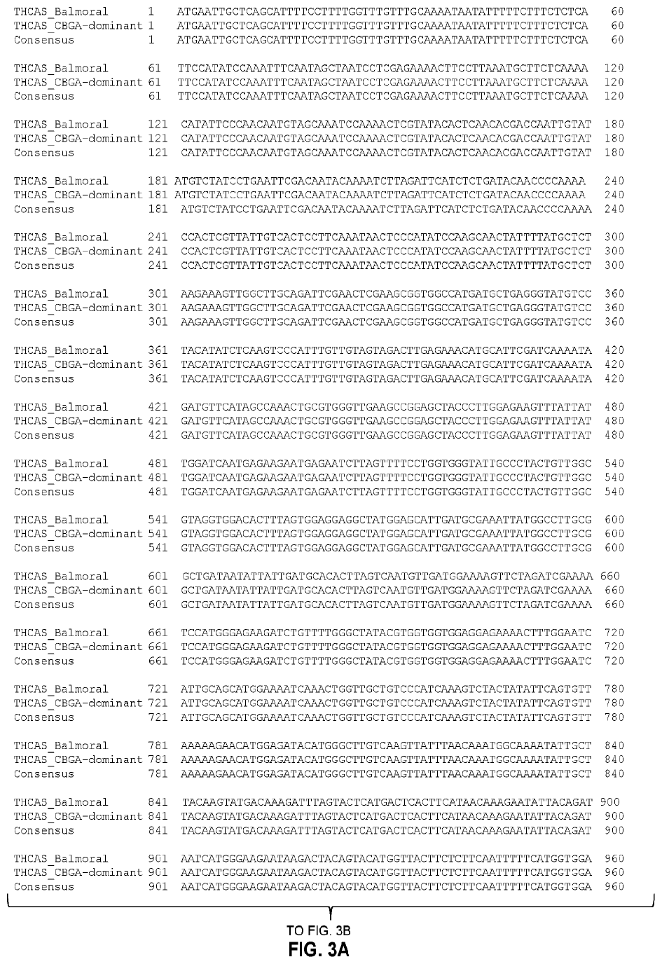

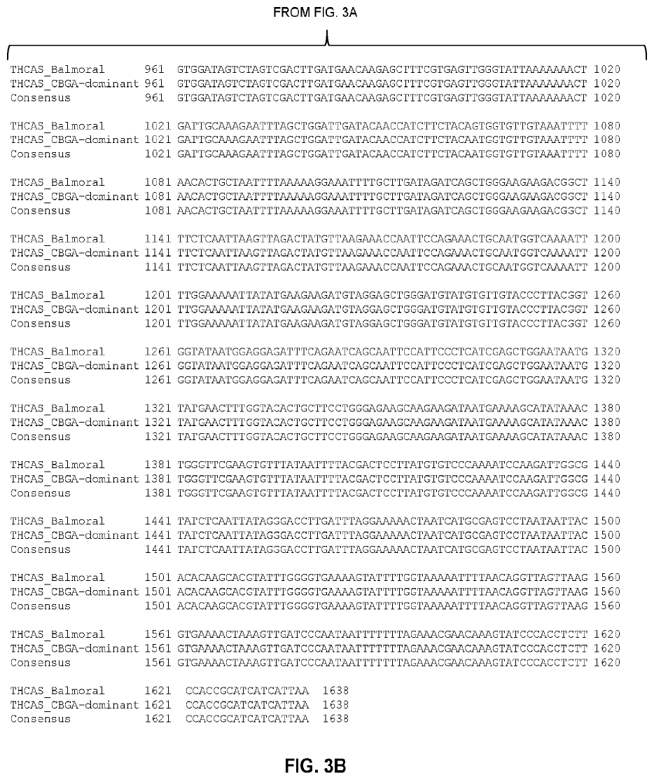

FIG. 3 shows a nucleic acid sequence alignment between THCA synthase isolated from a CBGA-dominant Cannabis plant and the THCAS extracted from Balmoral®. THCAS sequence from the CBGA-dominant Cannabis plant shows the G1064A single-nucleotide polymorphism (SNP) when aligned against the only THCAS sequence found in Balmoral®. A BLAST search found no other THCAS with G1064A SNP in any deposited sequence on the National Center for Biotechnology Information database.

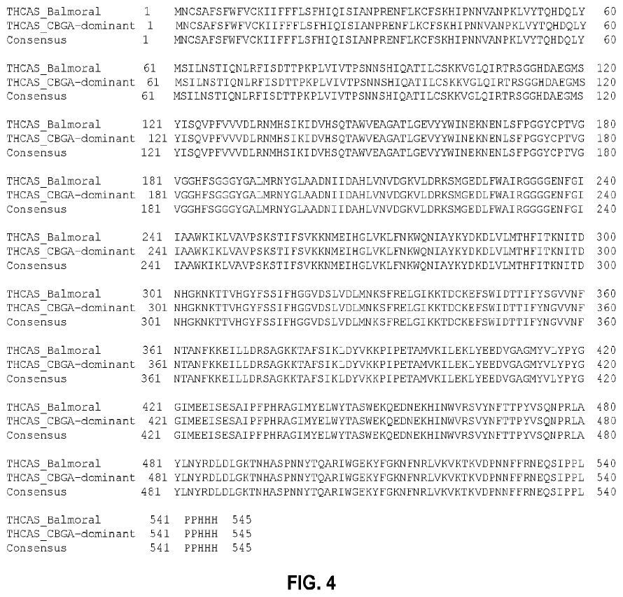

FIG. 4 shows alignment of the translated THCAS^{G1064A} nucleic acid sequence against the translated THCAS sequence from Balmoral®. It was observed that the G1064A SNP found in the CBGA-dominant Cannabis plant leads to an amino acid change between amino acid position 330 and amino acid position 360, specifically to a Serine to Asparagine (S to N) change at position 355.

A functional THCAS protein has been mapped by X-ray crystallography. THCAS protein was expressed in Spodoptera frugiperda Sf9 insect cells (Sirikantaramas et al., 2004) and subsequently purified into crystals suitable for X-ray diffraction from this system (Shoyama et al., 2005), allowing a tertiary structure with 2.75A resolution bound to flavin adenine dinucleotide (FAD) cofactor to be produced (Shoyama et al., 2012). Based on this model, S355 is positioned at the turn of two α-helices which is internal to the protein and faces the active site. The S355 side chain faces away from the active site and the amino acid makes three hydrogen bonds with other residues: S355 peptide N—H to F353 peptide C═O, S355 peptide C═O to V357 peptide N—H, and S355 side chain O—H to I352 peptide C═O. S355 is also adjacent to and within 5A of Cys 176, a crucial active site residue that is one of two amino acids covalently bound to the essential FAD cofactor necessary for THCAS catalytic activity.

The experimental data obtained in the context of the present technology suggests that S355N causes a shift in the position of Tyr354 and in the position of Tyr175 which are both putatively involved in substrate binding/selectivity.

The adjacent amino acid Y354 side chain faces towards the active site and is considered an essential active site residue, but likely to play a role in CBGA binding or specificity rather than a residue with direct catalytic activity (Shoyama et al., 2012).

Molecular docking models with CBGA as a substrate also support the function of Y354 and the positioning of the loop which S355 resides in CBGA binding (Alaoui et al., 2014). The S355 side chain hydrogen bond is predicted to be abolished by substitution with N at position 355, which could be expected to alter the positioning of the loop and active site residue Y354 and potentially block CBGA binding or misposition the FAD cofactor in the active site. N-linked glycosylation, the covalent addition of complex sugar groups to an asparagine residue, is a common modification to proteins. Typically, N-glycosylation occurs at the consensus sequence motif N-X-S/T where X can be any amino acid except proline (Aebi, 2013). THCAS has been confirmed to be N-glycosylated at N89, N168, N329, N467, N499 (Shoyama et al., 2012) which follow the N-X-S/T sequon motif. N-glycosylation has also been demonstrated in plants at non-consensus sequons N-X-C, N-G-X, and N-X-V (Aebi, 2013). A change of S355N may therefore lead to N-glycosylation at this site because it satisfies both N-G-X and N-X-V motifs. Based on its position in the crystal structure, glycosylation of N355 would be expected to disrupt proper protein folding and catalytic function.

### Example 2—Assessment of Cannabinoid Content in Homozygous THCAS^{G1064A} Cannabis Plant

To assess cannabinoid content in a homozygous THCAS^{G1064A} Cannabis plant, clonal meristem explants (with 2-4 expanded leaves) were cut from a CBGA-dominant mother plant bearing the THCAS G1064A polymorphism and rooted in rockwool cubes (Grodan®). Once clones had developed roots through the rockwool cubes they were transferred into 3-gallon deep water culture set ups with constant aeration from an aquarium pump and diffusion stone. Plants were grown for two weeks under Ray44 88W lights (Fluence®) under 18-hour light/6-hour dark cycle before being transferred to a mylar-lined growing chamber with Viparspectra 600W (Viparspectra®) lights to induce flowering under 12 hour light/12 hour dark cycle. Hydroponic vegetative stage nutrients Foliage pro 9:3:6 (Dyna-Gro®) were used at 1-2tsp/gallon with antimicrobial Clear Rez (EZClone®) being supplemented at ˜10 ml/gallon and then pH adjusted to 5.8. Flowering stage nutrients Bloom 3:12:6 (Dyna-Gro®) were used at 1-2 tsp/gallon with antimicrobial Clear Rez (EZClone®) being supplemented at ˜10 ml/gallon and then pH adjusted to 6. Temperature was maintained at around 22° C. and relative humidity was around 30%.

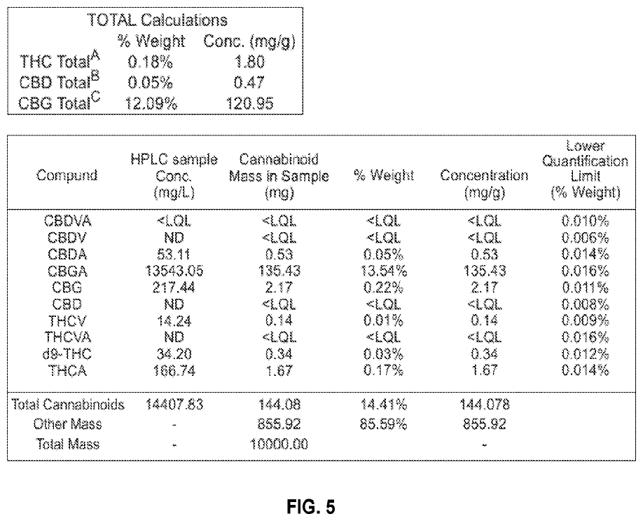

Harvested flower was dried on a rack at 30% humidity, ambient temperature, for one week ˜1 g of flower sample was ground and weighed and solvated into 10 mL of ethanol within a glass scintillation vial and sonicated for 30 minutes. The sample solution was filtered through a 0.22 μM nylon filter and then diluted 10 and 200-fold for HPLC analysis in 80/20 acetonitrile/isopropyl alcohol. Separation of 13 cannabinoids was achieved using a Raptor ARC-C18, 2.7 μM, 150×4.6 mM (Restek®) column with a gradient mobile phase consisting of A) water with 0.1% formic acid and B) acetonitrile+0.1% formic acid at a constant flow rate of 0.75 mL/min. The mobile phase composition started at 74% B, ramped to 78% B over 6 minutes, then ramped to 86% B between 6.01-10 minutes, held at 95% B from 10.01-11.50 minutes, and then returned to starting conditions at 11.51 (74% B) for 3.5 minutes. The column compartment was held at 4° C. while the autosampler remained at room temperature. 10 μL of each standard and sample was injected. A DAD (diode array detector) was employed for quantification of analytes using the 220 nm signal (no reference) as output. The R2 values of all calibration curves >0.995 and quantification of analytes from the CVS (calibration verification standard injected ˜10 injections) was within 10% the expected value. Total cannabinoid percentages were calculated as % weight neutral cannabinoid+(% weight acid cannabinoid*0.877). The results are presented in FIG. 5.

To assess cannabinoid content in a homozygous wild-type THCAS^{1064G/1064G} Cannabis plant, clonal plants propagated from the THCA-dominant medicinal cannabis plant Balmoral® were grown, flowered and harvested. During flowering, temperature was maintained at around 22-23° C. and relative humidity was around 40%. Flower was harvested at maturity and dried on a rack at around 30% humidity, around 16° C., for around two weeks. CBGA, CBG, CBDA, CBD, THCA, and THC levels of dried flower were measured by high performance liquid chromatography.

FIG. 5 shows the Cannabinoid profile of homozygous THCAS^{G1064A} Cannabis plant at 7 weeks post-flowering initiation. Table 1 presents chemotype data determined from various harvests of homozygous wild type THCAS Cannabis plant Balmoral®. The “decarboxylated bud” represents total cannabinoid fractions (without acidic fractions), whereas the “cured/dried bud” includes have acidic fractions. The homozygous THCAS^{G1064A} Cannabis plant exhibited an increase in CBGA concentration compared to the CBGA concentration of homozygous wild type THCAS^{1064G} Cannabis plant.

TABLE 1
Chemotype of a wild type THCAS Cannabis strain

| | CBD | CBDA | d9THC | d9THCA | CBG | CBGA |
|---|---|---|---|---|---|---|
| Description | (%) | (%) | (%) | (%) | (%) | (%) |
| Decarboxylated bud | 0.08 | | 18.32 | 0.1 | 0.22 | |
| Decarboxylated bud | 0.08 | | 17.66 | 0.09 | 0.21 | |
| Decarboxylated bud | 0.09 | | 19.15 | 0.08 | 0.22 | |
| Decarboxylated bud | 0.09 | | 19.32 | 0.09 | 0.24 | |
| Decarboxylated bud | 0.08 | | 18.08 | 0.07 | 0.22 | |
| Decarboxylated bud | 0.08 | | 18.89 | 0.09 | 0.22 | |
| Decarboxylated bud | 0.08 | | 18.1 | 0.08 | 0.21 | |
| Decarboxylated bud | 0.08 | | 17.9 | 0.11 | 0.2 | |
| Decarboxylated bud | 0.13 | | 20.18 | | 0.22 | 0.02 |
| Decarboxylated bud | 0.14 | | 21.13 | 0.08 | 0.21 | 0.01 |
| Decarboxylated bud | 0.13 | | 20.06 | | 0.22 | 0.02 |
| Decarboxylated bud | 0.13 | | 20.35 | 0.11 | 0.22 | 0.03 |
| Decarboxylated bud | 0.1 | | 19.889 | 0.015 | 0.176 | |
| Decarboxylated bud | 0.102 | | 21.36 | 0.04 | 0.168 | |
| Decarboxylated bud | 0.085 | | 17.591 | 0.016 | 0.158 | |
| Decarboxylated bud | 0.104 | | 21.634 | 0.078 | 0.175 | |
| Decarboxylated bud | 0.148 | | 21.787 | 0.038 | 0.201 | |
| Decarboxylated bud | 0.145 | | 21.284 | 0.017 | 0.208 | |
| Decarboxylated bud | 0.142 | | 20.805 | 0.064 | 0.204 | |
| Decarboxylated bud | 0.137 | | 19.652 | 0.01 | 0.206 | |
| Decarboxylated bud | 0.146 | | 21.147 | 0.066 | 0.204 | |
| Decarboxylated bud | 0.137 | | 20.41 | 0.03 | 0.196 | |
| Decarboxylated bud | 0.115 | | 19.318 | 0.045 | 0.191 | |
| Cured bud | | 0.09 | 0.82 | 22.97 | 0.07 | 0.21 |
| Cured bud | | 0.08 | 0.87 | 22.24 | 0.09 | 0.25 |
| Cured bud | | 0.08 | 0.87 | 21.77 | 0.1 | 0.22 |
| Cured bud | | 0.08 | 0.8 | 20.99 | 0.09 | 0.19 |
| Cured bud | | 0.08 | 0.78 | 22.16 | 0.07 | 0.23 |
| Cured bud | | 0.08 | 0.81 | 21.67 | 0.08 | 0.22 |
| Cured bud | | 0.08 | 0.79 | 20.14 | 0.06 | 0.22 |
| Cured bud | | 0.08 | 0.84 | 22.69 | 0.05 | 0.26 |
| Cured bud | | 0.08 | 0.88 | 22.32 | 0.1 | 0.2 |
| Cured bud | | 0.08 | 0.85 | 21.55 | 0.07 | 0.19 |

### Example 3—Performing THCAS Allele-Specific PCR

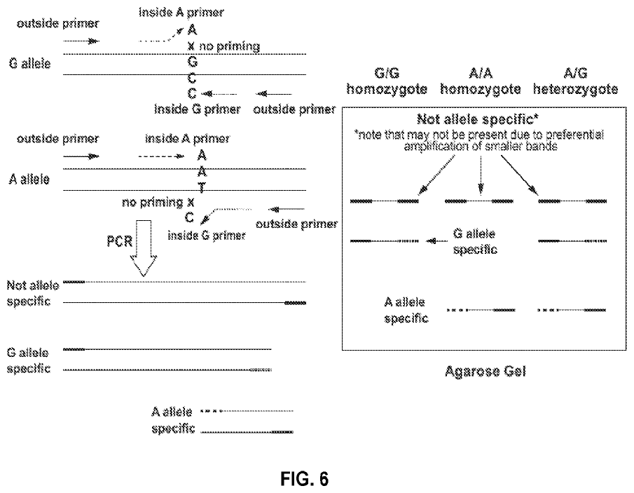

Young leaf tissue (20 mg) was collected from a THCAS^{1064G/1064G} Cannabis plant, from a THCAS^{1064A/1064A} Cannabis plant as well as from a THCAS^{1064G/1064A} Cannabis plant. The leaf tissues were homogenized by bead beating. Genomic DNA was extracted using a Maxwell® RSC Plant DNA Kit and Maxwell® RSC instrument (Promega®) using manufacturer's instructions. Allele-specific PCR was performed on genomic DNA template using GoTaq enzyme (Promega) and primers: THCAS forward primer: 5′-ATGAATTGCTCAGCATTT-3′ (SEQ ID NO: 9); THCAS reverse primer: 5′-AAAGATAATTAATGATGATGCG-3′ (SEQ ID NO: 10); THCAS^{1064G}-specific internal reverse primer: 5′-TGTTAAAATTTACAACACGAC-3′ (SEQ ID NO: 11); and THCAS^{1064A}-specific internal forward primer 5′-GATACAACCATCTTCTAGAA-3′ (SEQ ID NO: 12), based on the tetra-primer allele refractory mutation system (Ye et al., 1992) (FIG. 6) using manufacturer's instructions with the following thermal cycler program: 1) 95° C. for 2 min; 2) 95° C. for 30 sec; 3) 48° C. for 30 sec; 4) 72° C. for 2 min (repeat 2-4 29X); 5) 72° C. for 5 min; and 6) Hold 4° C.

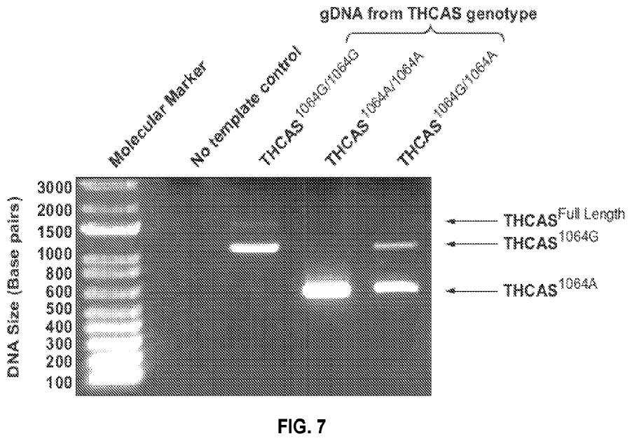

A PCR assay was performed to detect THCAS^{1064G} and THCAS^{1064A} alleles. The PCR products were separated on a 1% agarose gel and visualized with SYBR™ Safe DNA gel stain (ThermoFisher®). FIG. 7 shows the results of the PCR assay. A strong ˜600 base pair band was observed for the THCAS^{1064A/1064A} plant, indicating the presence of the SNP in both alleles. Amplification of the THCAS^{1064G/1064A} plant showed a strong ˜600 base pair band corresponding to the THCAS^{1064A} allele as well as a strong 1050 base pair band specific to the THCAS^{1064G} allele. Amplification of the THCAS^{1064G/1064G} plant only showed a strong 1050 base pair band specific to the THCAS^{1064G} allele. A 1635 base pair band is specific to the full-length amplification product possible in both alleles.

### Example 4—Assessing Segregation of the THCAS^{1064A} Allele by F1 Cross

To assess segregation of the THCAS^{1064A} allele, the CBGA-dominant plant homozygous for mutant THCAS (THCAS^{1064A/1064A}) which was characterized in FIGS. 2-5 and FIG. 7 was crossed with a CBDA-dominant cannabis plant capable of producing >10% CBDA content by dry weight in mature flowers. The CBGA-dominant plant and CBDA-dominant plants were clonally propagated, established, and induced to flower as described above. Male flowers were induced on the genetically female CBGA-dominant plant using foliar silver thiosulphate sprays at the beginning of the flowering period (Lubell & Brand, 2018). Pollen from the CBGA-dominant plant was then used to fertilize a flowering CBDA-dominant Cannabis plant and F1 seeds from the cross were harvested at 8 weeks post-flower induction.

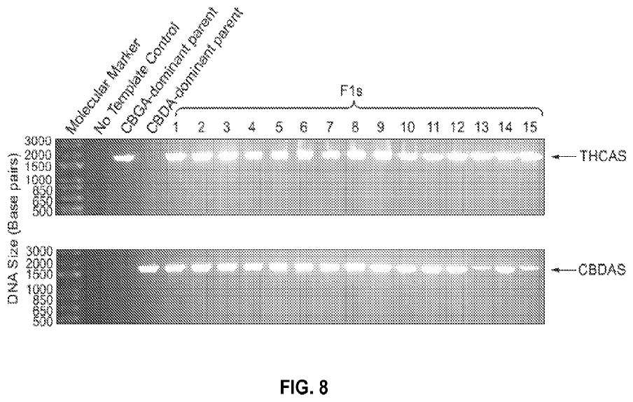

A number of 15 F1 seeds from the cross were germinated and established in rockwool (Grodan®) and young leaves were excised and used for gDNA extraction as described above. To investigate THCAS and CBDAS composition of the F1s, PCR was performed on F1 gDNA samples as well as gDNA isolated from both parental genotypes using primers SEQ ID NO: 5 and SEQ ID NO: 6 for THCAS and SEQ ID NO: 7 and SEQ ID NO: 8 for CBDAS as described above (FIG. 8). Results showed again that the CBGA-dominant parent only contained THCAS but not CBDAS. Conversely, the CBDA-dominant parent only contained CBDAS and not THCAS, which is in agreement with research showing that CBDA-dominant cannabis lacks a complete THCAS sequence (Grassa et al., 2021). All F1 progeny amplified both THCAS and CBDAS, supporting the Bt/Bd allele segregation hypothesis whereby the CBGA-dominant parent contributed THCAS and the CBDA-dominant parent contributed CBDAS.

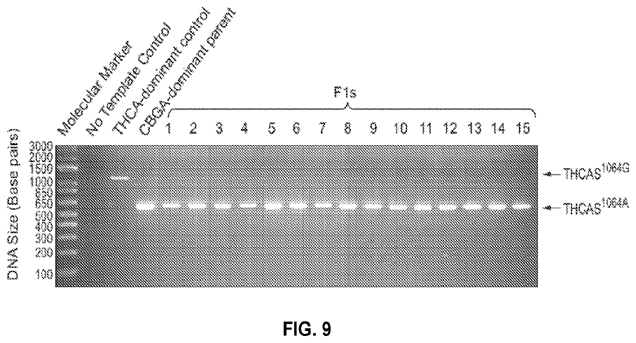

To discriminate between wild type THCAS^{1064G} and mutant THCAS^{1064A} alleles in the F1 population, allele-specific PCR was performed on gDNA samples from the F1s, the parental CBGA-dominant plant, and the control homozygous THCAS^{1064G/1064G} Balmoral® plant using primers SEQ ID NOs: 9-12 as described above. Results showed again that the CBGA-dominant plant was homozygous for the THCAS^{1064A} allele, and further showed that all F1 progeny only inherited the mutant THCAS^{1064A} allele. Together, the results depicted in FIG. 8 and FIG. 9 show that all F1 progeny inherited CBDAS from the CBDA-dominant parent and specifically the mutant THCAS^{1064A} allele from the CBGA-dominant parent.

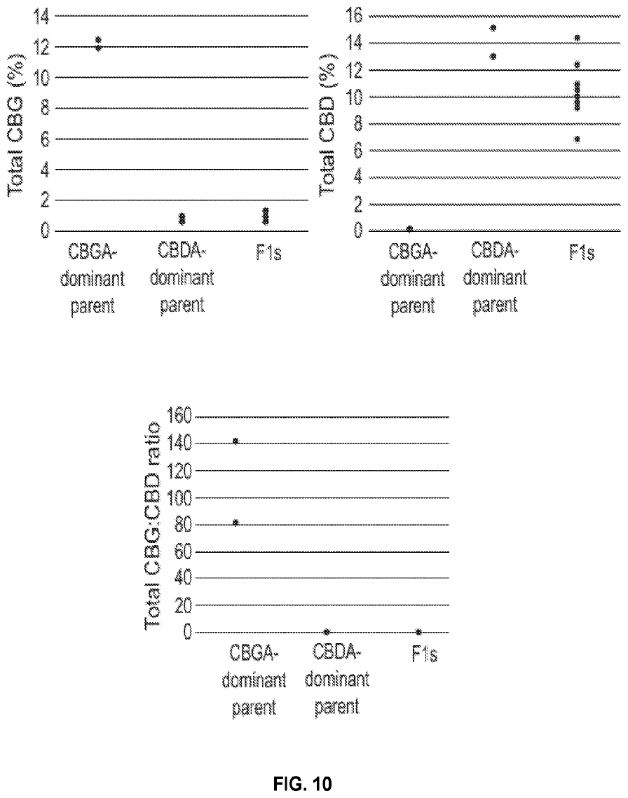

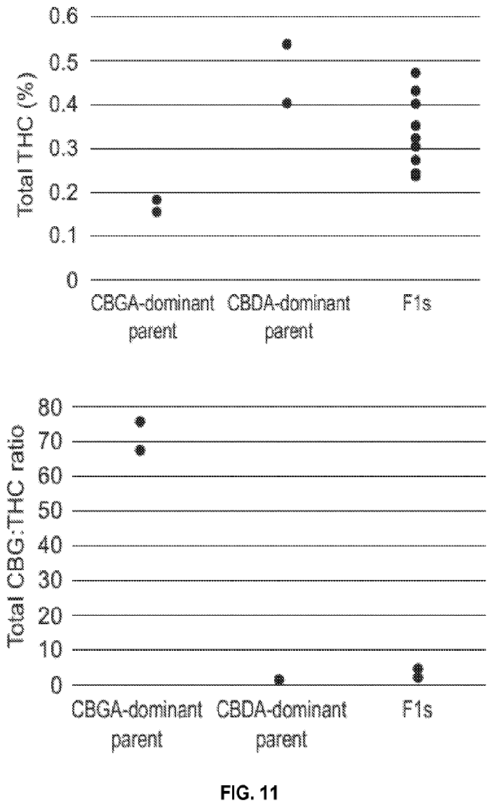

To further test if the THCAS^{1064A} allele is non-functional, 12 of the F1 progeny from the CBGA-dominant and CBDA-dominant plant cross as well as 2 replicate plants of each parent were clonally propagated, established, and flowered as described above. Flowers were harvested at 7 weeks post flower induction, prepared, and cannabinoid content was analyzed via HPLC as described above. Results for total cannabinoid content by dry weight in flowers for CBG, CBD, and CBG:CBD ratios are depicted in FIG. 10. Results for total cannabinoid content by dry weight in flowers for THC and CBG:THC ratios are depicted in FIG. 11. Each dot in FIG. 10 and FIG. 11 represents a data point from an individual plant.

All F1 progeny were CBDA-dominant and contained low total CBG content similar to the CBDA-dominant parent, with total CBG:CBD ratios no higher than 0.2 (FIG. 10). Conversely, the CBGA-dominant parent had total CBG:CBD ratios >100-fold higher than all other plants.

Together, the data clearly shows the CBGA-dominance observed in the parent is a recessive chemotype. Further this suggests the mutant THCAS^{1064A} allele is a non-functional THCAS and is recessive to CBDAS.

All F1 progeny contained low amounts of total THC concentrations similar to the CBDA-dominant parent and had similarly low total CBG:THC ratios compared the CBDA-dominant parent (FIG. 11). Low total THC content below 0.6% by dry weight in mature flowers of the CBDA-dominant parent and F1 progeny are consistent with observation that CBDAS non-specifically produces THCA from CBGA in low abundance (Zirpel et al., 2018). Collectively, the results from the F1 cross between the CBGA-dominant plant homozygous for mutant THCAS (THCAS^{1064A/1064A}) and a CBDA-dominant plant show that the mutant THCAS^{1064A} allele is a non-functional THCAS.

### Example 5—Assessing Segregation of THCAS^{1064A} Allele by Self-Pollination of F1 Parent

To further assess segregation of the THCAS^{1064A} allele, a CBDA-dominant F1 parent plant heterozygous for mutant THCAS (THCAS^{1064A}) and CBDAS that was characterized in FIGS. 8-11 was self-pollenated by male induction using silver thiosulphate sprays and selfed (S is of F1 parent) seeds were harvested as described above.

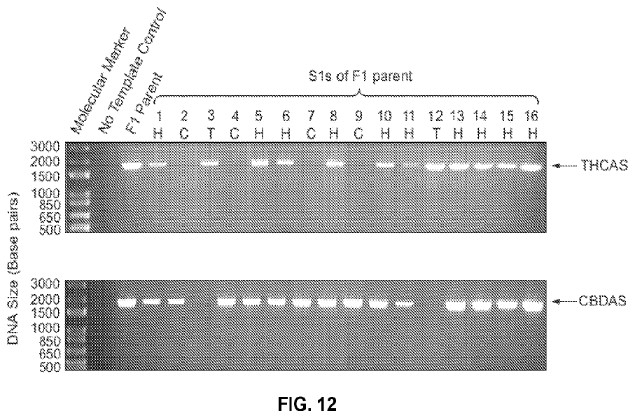

A number of 16 S1 seeds from the cross were germinated and established in rockwool (Grodan®) and young leaves were excised and used for gDNA extraction as described above. To investigate THCAS and CBDAS composition of the S1s, PCR was performed on S1 gDNA samples as well as gDNA isolated from the F1 parent using primers SEQ ID NO: 5 and SEQ ID NO: 6 for THCAS and SEQ ID NO: 7 and SEQ ID NO: 8 for CBDAS as described above (FIG. 12). Results of the S1 progeny showed that THCAS and CBDAS allele segregation followed the expected Bt/Bd Mendelian inheritance ratio from a heterozygous parent of 1:2:1 THCAS homozygous: THCAS/CBDAS heterozygous: CBDAS homozygous progeny. Specifically from the 16 S1 progeny, 2 were THCAS homozygous, 10 were THCAS/CBDAS heterozygous, and 4 were CBDAS homozygous yielding a Chi-squared test of independence value of 0.47 against an expected 4:8:4 distribution using the CHISQ.TEST function in Microsoft Excel (Table 2). This showed that THCAS and CBDAS inheritance followed the Bt/Bd allele hypothesis in the S1 progeny.

TABLE 2
Expected and actual distribution and Chi-squared test
value of THCAS homozygous, THCAS/CBDAS
heterozygous, and CBDAS homozygous S1 progeny

| | Expected | Actual | Chi Squared Test |
|---|---|---|---|
| THCAS homozygous | 4 | 2 | 0.47 |
| Heterozygous | 8 | 10 | |
| CBDAS homozygous | 4 | 4 | |

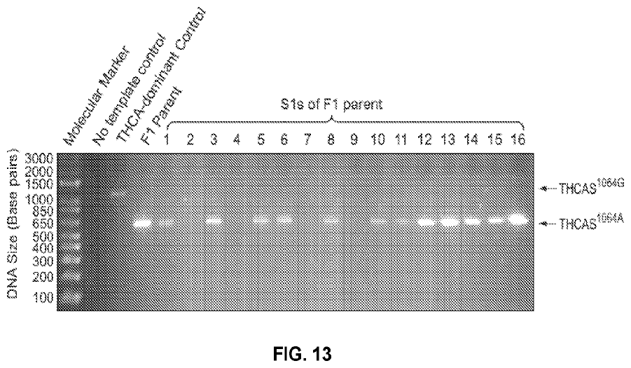

To discriminate between wild type THCAS^{1064G} and mutant THCAS^{1064A} alleles in the S1 population, allele-specific PCR was performed on gDNA samples from the S1 progeny, the F1 parental CBDA-dominant plant, and the control homozygous THCAS^{1064G/1064G} Balmoral® plant using primers SEQ ID NOs: 9-12 as described above (FIG. 13). Results showed again that the F1 parent and all S1 progeny positive for a THCAS allele identified in FIG. 12 only amplified the THCAS^{1064A} allele, showing that all S1 progeny only inherited the mutant THCAS^{1064A} allele.

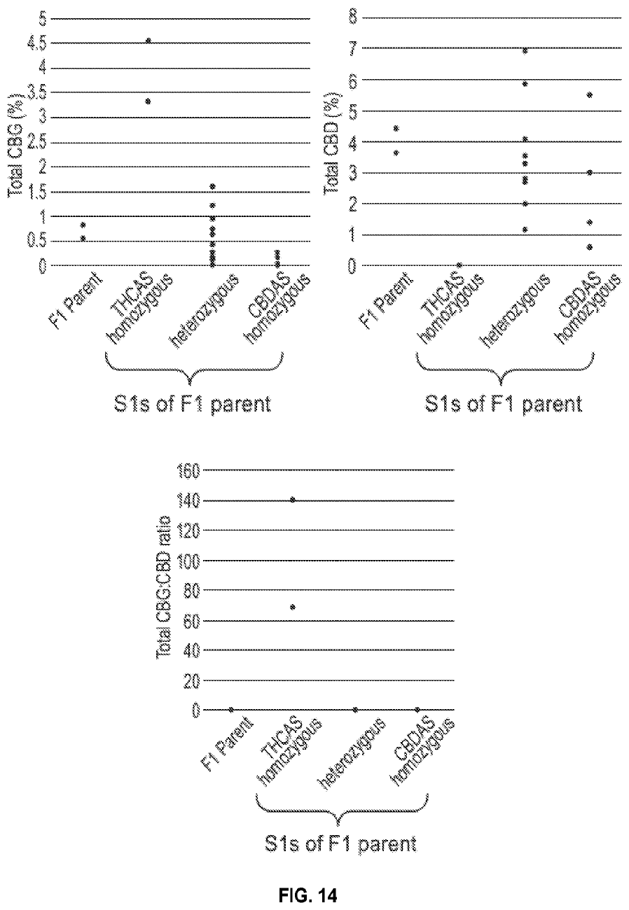

To further test if the THCAS^{1064A} allele is non-functional, all 16 of the S1 progeny from the CBDA-dominant F1 parent and 2 replicate plants of the parent were clonally propagated, established, and flowered as described above. Flowers were harvested early at 4 weeks post flower induction, prepared, and cannabinoid content was analyzed via HPLC as described above. Results for total cannabinoid content by dry weight in flowers for CBG, CBD, and CBG:CBD ratios are depicted in FIGS. 14 and S1 progeny were categorized into THCAS homozygous, THCAS/CBDAS heterozygous, and CBDAS homozygous based on allele screening results of FIG. 12. Each dot in FIG. 14 represents a data point from an individual plant.

The F1 parent and S1 progeny that were either CBDAS/THCAS heterozygous or CBDAS homozygous were CBDA-dominant and contained low total CBG content, with total CBG:CBD ratios no higher than 0.3 (FIG. 14). Conversely, the two S1 progeny that were THCAS homozygous for the mutant allele (THCAS^{1064A/1064A}) were CBGA-dominant, contained only trace amounts of total CBD, and had total CBG:CBD ratios >100-fold higher than all other plants.

Together, the data from the S1 progeny from a CBDA-dominant F parent plant heterozygous for mutant THCAS (THCAS^{1064A}) and CBDAS clearly shows CBGA-dominance is a recessive chemotype due to homozygosity of the mutant THCAS allele (THCAS^{1064A/1064A}). Further this shows the mutant THCAS^{1064A} allele is a non-functional THCAS and is recessive to CBDAS.

### INCORPORATION BY REFERENCE

All references cited in this specification, and their references, are incorporated by reference herein in their entirety where appropriate for teachings of additional or alternative details, features, and/or technical background.

### EQUIVALENTS

While the disclosure has been particularly shown and described with reference to particular embodiments, it will be appreciated that variations of the above-disclosed and other features and functions, or alternatives thereof, may be desirably combined into many other different systems or applications. Also, that various presently unforeseen or unanticipated alternatives, modifications, variations or improvements therein may be subsequently made by those skilled in the art which are also intended to be encompassed by the following embodiments.

### BIBLIOGRAPHY

Aebi, M. (2013). N-linked protein glycosylation in the ER. In Biochimica et Biophysica Acta—Molecular Cell Research. https://doi.org/10.1016/j.bbamcr.2013.04.001
Alaoui, M., Ibrahimi, A., Semlali, O., Tarhda, Z., Marouane, M., Najwa, A., Soulaymani, A., & Fahime, E. El. (2014) Affinity comparison of different THCA synthase to CBGA using modeling computational approaches. Bioinformation. https://doi.org/10.6026/97320630010033
Borrelli, F., Fasolino, I., Romano, B., Capasso, R., Maiello, F., Coppola, D., Orlando, P., Battista, G., Pagano, E., Di Marzo, V., & Izzo, A. A. (2013). Beneficial effect of the non-psychotropic plant cannabinoid cannabigerol on experimental inflammatory bowel disease. Biochemical Pharmacology. https://doi.org/10.1016/j.bcp.2013.01.017
Borrelli, F., Pagano, E., Romano, B., Panzera, S., Maiello, F., Coppola, D., De Petrocellis, L., Buono, L., Orlando, P., & Izzo, A. A. (2014). Colon carcinogenesis is inhibited by the TRPM8 antagonist cannabigerol, a Cannabis-derived non-psychotropic cannabinoid. Carcinogenesis. https://doi.org/10.1093/carcin/bgu205
Brierley, D. I., Samuels, J., Duncan, M., Whalley, B. J., & Williams, C. M. (2016). Cannabigerol is a novel, well-tolerated appetite stimulant in pre-satiated rats. Psychopharmacology. https://doi.org/10.1007/s00213-016-4397-4
Colasanti, B. K., Craig, C. R., & Allara, R. D. (1984). Intraocular pressure, ocular toxicity and neurotoxicity after administration of cannabinol or cannabigerol. Experimental Eye Research. https://doi.org/10.1016/0014-4835(84)90013-7
De Meijer, E. P. M., & Hammond, K. M. (2005). The inheritance of chemical phenotype in Cannabis sativa L. (II): Cannabigerol predominant plants. Euphytica. https://doi.org/10.1007/s10681-005-1164-8
De Meijer, Etienne P. M., Bagatta, M., Carboni, A., Crucitti, P., Moliterni, V. M. C., Ranalli, P., & Mandolino, G. (2003). The inheritance of chemical phenotype in Cannabis sativa L. Genetics.
Di Marzo, V., & Piscitelli, F. (2015). The Endocannabinoid System and its Modulation by Phytocannabinoids. In Neurotherapeutics. https://doi.org/10.1007/s13311-015-0374-6
ElSohly, M. A., & Slade, D. (2005). Chemical constituents of marijuana: The complex mixture of natural cannabinoids. Life Sciences. https://doi.org/10.1016/j.1fs.2005.09.011
Fournier, G., Richez-Dumanois, C., Duvezin, J., Mathieu, J. P., & Paris, M. (1987). Identification of a new chemotype in Cannabis sativa: Cannabigerol-dominant plants, biogenetic and agronomic prospects. Planta Medica. https://doi.org/10.1055/s-2006-962705
Gagne, S. J., Stout, J. M., Liu, E., Boubakir, Z., Clark, S. M., & Page, J. E. (2012). Identification of olivetolic acid cyclase from Cannabis sativa reveals a unique catalytic route to plant polyketides. Proceedings of the National Academy of Sciences of the United States of America. https://doi.org/10.1073/pnas.1200330109
Gugliandolo, A., Pollastro, F., Grassi, G., Bramanti, P., & Mazzon, E. (2018). In vitro model of neuroinflammation: Efficacy of cannabigerol, a non-psychoactive cannabinoid. International Journal of Molecular Sciences. https://doi.org/10.3390/ijms19071992
Laverty, K. U., Stout, J. M., Sullivan, M. J., Shah, H., Gill, N., Holbrook, L., Deikus, G., Sebra, R., Hughes, T. R., Page, J. E., & Van Bakel, H. (2019). A physical and genetic map of Cannabis sativa identifies extensive rearrangements at the THC/CBD acid synthase loci. Genome Research. https://doi.org/10.1101/gr.242594.118
Lewis, M. A., Russo, E. B., & Smith, K. M. (2018). Pharmacological Foundations of Cannabis Chemovars. Planta Medica. https://doi.org/10.1055/s-0043-122240
McPartland, J. M., & Small, E. (2020). A classification of endangered high-THC Cannabis (Cannabis sativa subsp. indica) domesticates and their wild relatives. PhytoKeys. https://doi.org/10.3897/PHYTOKEYS.144.46700
Onofri, C., De Meijer, E. P. M., & Mandolino, G. (2015). Sequence heterogeneity of cannabidiolic- and tetrahydrocannabinolic acid-synthase in Cannabis sativa L. and its relationship with chemical phenotype. Phytochemistry. https://doi.org/10.1016/j.phytochem2015.03.006
Pacifico, D., Miselli, F., Micheler, M., Carboni, A., Ranalli, P., & Mandolino, G. (2006). Genetics and marker-assisted selection of the chemotype in Cannabis sativa L. Molecular Breeding https://doi.org/10.1007/s11032-005-5681-x
Shoyama, Y., Takeuchi, A., Taura, F., Tamada, T., Adachi, M., Kuroki, R., Shoyama, Y., & Morimoto, S. (2005). Crystallization of Δ1-tetrahydrocannabinolic acid (THCA) synthase from Cannabis sativa. Acta Crystallographica Section F: Structural Biology and Crystallization Communications. https://doi.org/10.1107/S1744309105023365
Shoyama, Y., Tamada, T., Kurihara, K., Takeuchi, A., Taura, F., Arai, S., Blaber, M., Shoyama, Y., Morimoto, S., & Kuroki, R. (2012). Structure and function of Δ1-tetrahydrocannabinolic acid (THCA) synthase, the enzyme controlling the psychoactivity of Cannabis sativa. Journal of Molecular Biology. https://doi.org/10.1016/j.jmb.2012.06.030
Sirikantaramas, S., Morimoto, S., Shoyama, Y., Ishikawa, Y., Wada, Y., Shoyama, Y., & Taura, F. (2004). The gene controlling marijuana psychoactivity. Molecular cloning and heterologous expression of Δ1-tetrahydrocannabinolic acid synthase from Cannabis sativa L. Journal of Biological Chemistry. https://doi.org/10.1074/jbc.M403693200
Small, E., & Beckstead, H. D. (1973). Cannabinoid phenotypes in Cannabis sativa. Nature. https://doi.org/10.1038/245147a0
Valdeolivas, S., Navarrete, C., Cantarero, I., Bellido, M. L., Muñoz, E., & Sagredo, O. (2015). Neuroprotective Properties of Cannabigerol in Huntington's Disease: Studies in R6/2 Mice and 3-Nitropropionate-lesioned Mice. Neurotherapeutics https://doi.org/10.1007/s13311-014-0304-z
van Bakel, H., Stout, J. M., Cote, A. G., Tallon, C. M., Sharpe, A. G., Hughes, T. R., & Page, J. E. (2011). The draft genome and transcriptome of Cannabis sativa. Genome Biology. https://doi.org/10.1186/gb-2011-12-10-r102
Yang, R., Berthold, E., McCurdy, C. R., da Silva Benevenute, S., Brym, Z. T., & Freeman, J. H. (2020).
Development of Cannabinoids in Flowers of Industrial Hemp (Cannabis sativa L.)—a Pilot Study. Journal of Agricultural and Food Chemistry. https://doi.org/10.1021/acs.jafe.0c01211
Grassa, C. J., Wenger, J. P., Dabney, C., Poplawski, S. G., Motley, S. T., Michael, T. P., Schwartz, C. J., & Weiblen, G. D. (2018). A complete Cannabis chromosome assembly and adaptive admixture for elevated cannabidiol (CBD) content. BioRxiv. https://doi.org/10.1101/458083
Laverty, K. U., Stout, J. M., Sullivan, M. J., Shah, H., Gill, N., Holbrook, L., Deikus, G., Sebra, R., Hughes, T. R., Page, J. E., & Van Bakel, H. (2019). A physical and genetic map of Cannabis sativa identifies extensive rearrangements at the THC/CBD acid synthase loci. Genome Research. https://doi.org/10.1101/gr.242594.118
McPartland, J. M., & Guy, G. W. (2017). Models of Cannabis Taxonomy, Cultural Bias, and Conflicts between Scientific and Vernacular Names. Botanical Review. https://doi.org/10.1007/s12229-017-9187-0
Onofri, C., De Meijer, E. P. M., & Mandolino, G. (2015). Sequence heterogeneity of cannabidiolic- and tetrahydrocannabinolic acid-synthase in Cannabis sativa L. and its relationship with chemical phenotype. Phytochemistry. https://doi.org/10.1016/j.phytochem.2015.03.006
Russo, E. B. (2011). Taming THC: Potential cannabis synergy and phytocannabinoid-terpenoid entourage effects. In British Journal of Pharmacology. https://doi.org/10.1111/j.1476-5381.2011.01238.x
Schultes, R. E., Schultes, R. E., Klein, W. M., Plowman, T., & Lockwood, T. E. (1974). Cannabis: an example of taxonomic neglect. Botanical Museum Leaflets, Harvard University.
Small, E. (1972). Interfertility and chromosomal uniformity in Cannabis. Canadian Journal of Botany. https://doi.org/10.1139/b72-248
Small, E., & Cronquist, A. (1976). A PRACTICAL AND NATURAL TAXONOMY FOR CANNABIS. TAXON. https://doi.org/10.2307/1220524
Weiblen, G. D., Wenger, J. P., Craft, K. J., ElSohly, M. A., Mehmedic, Z., Treiber, E. L., & Marks, M. D. (2015). Gene duplication and divergence affecting drug content in Cannabis sativa. New Phytologist. https://doi.org/10.1111/nph.13562
Grassa, C. J., Weiblen, G. D., Wenger, J. P., Dabney, C., Poplawski, S. G., Timothy Motley, S., Michael, T. P., & Schwartz, C. J. (2021). A new Cannabis genome assembly associates elevated cannabidiol (CBD) with hemp introgressed into marijuana. New Phytologist. https://doi.org/10.1111/nph.17243
Lubell, J. D., & Brand, M. H. (2018). Foliar sprays of silver thiosulfate produce male flowers on female hemp plants. HortTechnology. https://doi.org/10.21273/HORTTECH04188-18
Zirpel, B., Kayser, O., & Stehle, F. (2018). Elucidation of structure-function relationship of THCA and CBDA synthase from Cannabis sativa L. Journal of Biotechnology. https://doi.org/10.1016/j.jbiotec.2018.07.031

1. The Cannabis plant of claim 48, wherein the Cannabis plant has a cannabigerol (CBG) content greater than about 5% by weight.

2.-13. (canceled)

14. An isolated nucleic acid molecule comprising an altered tetrahydrocannabinolic acid synthase (THCAS) allele, wherein the altered THCAS allele encodes for a minimally functional or non-functional THCAS enzyme and causes an increase in cannabigerolic acid (CBGA) and/or cannabigeriol (CBG) content.

15. The isolated nucleic acid molecule of claim 14, wherein the altered THCAS allele encodes for a minimally functional or non-functional THCAS enzyme that impedes conversion of CBGA to THCA.

16. The isolated nucleic acid molecule of claim 14, wherein the minimally functional or the non-functional THCAS enzyme comprises an activity-altering change.

17. The isolated nucleic acid of claim 16, wherein the activity-altering change is a small nucleotide polymorphism (SNP), wherein the SNP results in an amino acid change at position 355 of the encoded polypeptide.

18.-19. (canceled)

20. The isolated nucleic acid of claim 17, wherein the amino acid change at position 355 is a serine to asparagine change.

21. The isolated nucleic acid of claim 17, wherein the amino acid change at position 355 is a serine to glutamine.

22. The isolated nucleic acid molecule of claim 17, the isolated nucleic acid molecule comprising the nucleic acid sequence as set forth in SEQ ID NO: 2.

23.-47. (canceled)

48. A Cannabis plant comprising an altered tetrahydrocannabinolic acid (THCA) synthase allele, wherein the altered tetrahydrocannabinolic acid (THCA) synthase allele encodes a minimally functional or a non-functional tetrahydrocannabinolic acid (THCA) synthase that causes an increase in cannabigerolic acid (CBGA) and/or cannabigerol (CBG) levels.

49. (canceled)

50. The Cannabis plant of claim 48, wherein the activity-altering mutation results in an amino acid change at position 355 encoded THCA synthase.

51.-60. (canceled)

61. A method for increasing levels of cannabigerolic acid (CBGA) and/or cannabigerol (CBG) in a tissue or a cell, the method comprising introducing an activity-altering change in the nucleic acid sequence encoding for tetrahydrocannabinolic acid synthase (THCAS) in the tissue or the cell.

62. The method of claim 61, wherein the activity-altering change results in a THCAS allele that encodes for a minimally functional or non-functional THCAS that impedes conversion of CBGA to THCA.

63. The method of claim 62, wherein the activity-altering change is a small nucleotide polymorphism.

64. The method of claim 63, wherein the small nucleotide polymorphism results in an amino acid change between position 330 and 360 of the encoded polypeptide.

65. The method of claim 64, wherein the amino acid change is at position 355.

66. The method of claim 65, wherein the amino acid change at position 355 is a serine to asparagine change.

67. The method of claim 65, wherein the amino acid change at position 355 is a serine to glutamine.

68. The method of claim 61, comprising using an endonuclease enzyme targeting a nucleic acid sequence coding for THCAS and introducing the enzyme endonuclease enzyme into a genome of the tissue or cell.

69. The method of claim 68, wherein the endonuclease enzyme is a CRISPR/Cas system.

70. The method of claim 69, where the CRISPR/Cas system introduces an activity-altering change in the nucleic acid sequence coding for the THCAS.

71.-73. (canceled)


SEQUENCE LISTING (12 sequences)

SEQ ID NO: 1 - DNA, 1638 bp, Cannabis sativa
atgaattgctcagcattttccttttggtttgtttgcaaaataatatttttctttctctcattccatatccaaatttcaatagctaatcctcgagaaaacttccttaaatgcttctcaaaacatattcccaacaatgtagcaaatccaaaactcgtatacactcaacacgaccaattgtatatgtctatcctgaattcgacaatacaaaatcttagattcatctctgatacaaccccaaaaccactcgttattgtcactccttcaaataactcccatatccaagcaactattttatgctctaagaaagttggcttgcagattcgaactcgaagcggtggccatgatgctgagggtatgtcctacatatctcaagtcccatttgttgtagtagacttgagaaacatgcattcgatcaaaatagatgttcatagccaaactgcgtgggttgaagccggagctacccttggagaagtttattattggatcaatgagaagaatgagaatcttagttttcctggtgggtattgccctactgttggcgtaggtggacactttagtggaggaggctatggagcattgatgcgaaattatggccttgcggctgataatattattgatgcacacttagtcaatgttgatggaaaagttctagatcgaaaatccatgggagaagatctgttttgggctatacgtggtggtggaggagaaaactttggaatcattgcagcatggaaaatcaaactggttgctgtcccatcaaagtctactatattcagtgttaaaaagaacatggagatacatgggcttgtcaagttatttaacaaatggcaaaatattgcttacaagtatgacaaagatttagtactcatgactcacttcataacaaagaatattacagataatcatgggaagaataagactacagtacatggttacttctcttcaatttttcatggtggagtggatagtctagtcgacttgatgaacaagagctttcgtgagttgggtattaaaaaaactgattgcaaagaatttagctggattgatacaaccatcttctacagtggtgttgtaaattttaacactgctaattttaaaaaggaaattttgcttgatagatcagctgggaagaagacggctttctcaattaagttagactatgttaagaaaccaattccagaaactgcaatggtcaaaattttggaaaaattatatgaagaagatgtaggagctgggatgtatgtgttgtacccttacggtggtataatggaggagatttcagaatcagcaattccattccctcatcgagctggaataatgtatgaactttggtacactgcttcctgggagaagcaagaagataatgaaaagcatataaactgggttcgaagtgtttataattttacgactccttatgtgtcccaaaatccaagattggcgtatctcaattatagggaccttgatttaggaaaaactaatcatgcgagtcctaataattacacacaagcacgtatttggggtgaaaagtattttggtaaaaattttaacaggttagttaaggtgaaaactaaagttgatcccaataatttttttagaaacgaacaaagtatcccacctcttccaccgcatcatcattaa

SEQ ID NO: 2 - DNA, 1638 bp, Cannabis sativa
atgaattgctcagcattttccttttggtttgtttgcaaaataatatttttctttctctcattccatatccaaatttcaatagctaatcctcgagaaaacttccttaaatgcttctcaaaacatattcccaacaatgtagcaaatccaaaactcgtatacactcaacacgaccaattgtatatgtctatcctgaattcgacaatacaaaatcttagattcatctctgatacaaccccaaaaccactcgttattgtcactccttcaaataactcccatatccaagcaactattttatgctctaagaaagttggcttgcagattcgaactcgaagcggtggccatgatgctgagggtatgtcctacatatctcaagtcccatttgttgtagtagacttgagaaacatgcattcgatcaaaatagatgttcatagccaaactgcgtgggttgaagccggagctacccttggagaagtttattattggatcaatgagaagaatgagaatcttagttttcctggtgggtattgccctactgttggcgtaggtggacactttagtggaggaggctatggagcattgatgcgaaattatggccttgcggctgataatattattgatgcacacttagtcaatgttgatggaaaagttctagatcgaaaatccatgggagaagatctgttttgggctatacgtggtggtggaggagaaaactttggaatcattgcagcatggaaaatcaaactggttgctgtcccatcaaagtctactatattcagtgttaaaaagaacatggagatacatgggcttgtcaagttatttaacaaatggcaaaatattgcttacaagtatgacaaagatttagtactcatgactcacttcataacaaagaatattacagataatcatgggaagaataagactacagtacatggttacttctcttcaatttttcatggtggagtggatagtctagtcgacttgatgaacaagagctttcgtgagttgggtattaaaaaaactgattgcaaagaatttagctggattgatacaaccatcttctacaatggtgttgtaaattttaacactgctaattttaaaaaggaaattttgcttgatagatcagctgggaagaagacggctttctcaattaagttagactatgttaagaaaccaattccagaaactgcaatggtcaaaattttggaaaaattatatgaagaagatgtaggagctgggatgtatgtgttgtacccttacggtggtataatggaggagatttcagaatcagcaattccattccctcatcgagctggaataatgtatgaactttggtacactgcttcctgggagaagcaagaagataatgaaaagcatataaactgggttcgaagtgtttataattttacgactccttatgtgtcccaaaatccaagattggcgtatctcaattatagggaccttgatttaggaaaaactaatcatgcgagtcctaataattacacacaagcacgtatttggggtgaaaagtattttggtaaaaattttaacaggttagttaaggtgaaaactaaagttgatcccaataatttttttagaaacgaacaaagtatcccacctcttccaccgcatcatcattaa

SEQ ID NO: 3 - protein, 545 aa, Cannabis sativa
MNCSAFSFWFVCKIIFFFLSFHIQISIANPRENFLKCFSKHIPNNVANPKLVYTQHDQLYMSILNSTIQNLRFISDTTPKPLVIVTPSNNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFRELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFTTPYVSQNPRLAYLNYRDLDLGKTNHASPNNYTQARIWGEKYFGKNFNRLVKVKTKVDPNNFFRNEQSIPPLPPHHH

SEQ ID NO: 4 - protein, 545 aa, Cannabis sativa
MNCSAFSFWFVCKIIFFFLSFHIQISIANPRENFLKCFSKHIPNNVANPKLVYTQHDQLYMSILNSTIQNLRFISDTTPKPLVIVTPSNNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFRELGIKKTDCKEFSWIDTTIFYNGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFTTPYVSQNPRLAYLNYRDLDLGKTNHASPNNYTQARIWGEKYFGKNFNRLVKVKTKVDPNNFFRNEQSIPPLPPHHH

SEQ ID NO: 5 - DNA, 26 bp, Artificial Sequence
  Primer forward
tttatcacatgtgactatttaatgac

SEQ ID NO: 6 - DNA, 30 bp, Artificial Sequence
  Primer reverse
tgattaactaattgataagggaaatatatc

SEQ ID NO: 7 - DNA, 21 bp, Artificial Sequence
  Primer forward
ccctgctccaatatataaagc

SEQ ID NO: 8 - DNA, 19 bp, Artificial Sequence
  Primer reverse
atacacagtacatccggac

SEQ ID NO: 9 - DNA, 18 bp, Artificial Sequence
  Primer forward
atgaattgctcagcattt

SEQ ID NO: 10 - DNA, 22 bp, Artificial Sequence
  Primer reverse
aaagataattaatgatgatgcg

SEQ ID NO: 11 - DNA, 21 bp, Artificial Sequence
  Primer reverse
tgttaaaatttacaacacgac

SEQ ID NO: 12 - DNA, 20 bp, Artificial Sequence
  Primer reverse
gatacaaccatcttctagaa